(19) United States
(12) Patent Application Publication
(10) Pub. No.: US 2014/0308272 A1
(43) Pub. Date: Oct. 16, 2014

(54) TREATMENT OF PRION PROTEIN RELATED DISEASES

(71) Applicant: Sylvan Pharmaceuticals Pty, Ltd., Bondi Junction (AU)

(72) Inventors: John Fergus McEwan, Blakehurst (AU); David Cullis-Hill, Woollahra (AU); Martin Lyndale Windsor, Randwick (AU)

(73) Assignee: Sylvan Pharmaceuticals Pty, Ltd., Bondi Junction (AU)

(21) Appl. No.: 14/158,385

(22) Filed: Jan. 17, 2014

Related U.S. Application Data
(63) Continuation of application No. 12/672,737, filed on Nov. 15, 2010, now abandoned.

(30) Foreign Application Priority Data
Aug. 9, 2007 (AU) 2007904281

(51) Int. Cl. C07K 16/44

(52) U.S. Cl. CPC C07K 16/44
USPC 424/133.1; 424/175.1; 424/141.1; 435/332; 424/178.1; 514/61; 514/56; 514/1.1; 530/389.8; 530/388.9; 530/387.3; 536/18.7; 536/123.1; 536/21

(57) ABSTRACT

According to the present invention, treatment of prion protein related diseases, and in particular cancer tumours, is effected by providing to a subject in need thereof a therapeutically effective amount of an agent capable of modulating binding of a prion protein and/or a prion-like protein to a disease related GAG and/or HSPG.

This application is a continuation application of U.S. patent application Ser. No. 12/672,727, filed Nov. 15, 2010, which is a 371 application of PCT/AU2008/001155 filed Aug. 8, 2008, each of which is incorporated herein by reference.

### FIELD OF THE INVENTION

This invention relates to the treatment of prion protein-related diseases such as cancer. Applications of the invention also include amyloid diseases such as amyloidosis and Alzheimer's disease, as well as use in inflammatory conditions and transplant technology. This invention concerns glycosaminoglycans (GAGs), synthetic polysulfated polysaccharides, heparan sulphate proteoglycans (HSPGs) and pharmaceutical compositions comprising these, as well as antibodies or fragments of antibodies to GAGs, synthetic polysulfated polysaccharides, HSPGs, prion protein, doppel protein, and shadoo protein and pharmaceutical compositions thereof.

### BACKGROUND OF THE INVENTION

### 1. Background Art

Prion is an acronym for ‘proteinaceous infectious particle’. The term was coined in 1982 by Stanley B. Prusiner, a neurologist at the University of California at San Francisco, who proposed that a new type of pathogen consisting solely of protein is responsible for a group of deadly neurodegenerative diseases called Transmissible Spongiform Encephalopathies (TSEs). The diseases include for example scrapie in sheep, bovine spongiform encephalopathy (BSE or ‘mad cow disease’) in cattle and Creutzfeldt-Jakob Disease (CJD) in people.

Despite extensive research in prion disease and prion protein (PrP), the role of normal cellular prion protein (PrP^{c}) is still unknown. PrP^{c} is expressed widely in the immune system, in haematopoietic stem cells and mature lymphoid and myeloid compartments in addition to cells of the central nervous system. Several lines of evidence indicate that PrP^{c} may protect human neurons from various kinds of internal or environmental stress. Self-renewal of haematopoietic stem cells may require PrP^{c}, and PrP^{c} may also have a distinctive role in the immune system. Disruption of PrP^{c} can result in an aberrant regulation of a battery of genes that are important for cell proliferation, differentiation and survival. PrP^{c} seems to play a fundamental role in regulating cellular physiological functions within a multifaceted network.

### Prion Protein Diseases

The nature of TSE infectivity has remained elusive. Several lines of evidence suggest that the prion protein may not even be working alone. For example scientists have identified the importance of ‘co-factors’ such as RNA and sulfated glycosaminoglycans (GAGs) for infectious transmissibility. It has also been determined that PrP^{Sc} can be separated from infectivity, and that accumulation of PrP^{Sc} is not always associated with pathology.

PrP^{c} has been shown to be involved in several types of cancer where a function in apoptosis has been delineated. Significantly, PrP^{c} is over expressed in some cancer cell lines (Du et al, Int J Cancer 113 (2005) p 213). Also, PrP^{c} has been shown to bind metals and thus could be a copper-binding antioxidant in vivo. Aberrant trace metal levels (particularly manganese) were detected in the brains of patients with sporadic CJD, in support of this theory. Furthermore, it has been suggested that PrP^{c} forms part of a cellular antioxidant defense mechanism.

Expression of PrP in Alzheimer's disease has been noted: McNeill, A., MUM 8 (2004) p 7-14, and similarly, in acute signaling situations such as in organ transplant, upregulation of GAG/HSPG signaling rapidly takes place. This signaling leads to ischemia.

### Association Between PrP^{c} and HSPG

An association of PrP with sulfated glycosaminoglycans has been reported (Caughey et al, Ace. Chem. Res. 39 (2006) p 646). Indeed, the complex interaction between PrP and heparin/HS has been noted, along with its implications for the functions of PrP. Sulfated polysaccharides have been found to both inhibit and stimulate PrP^{Sc} formation. Pentosan polysulfate, for example, is an effective treatment for prion diseases (TSEs) (Larramendy-Gozalo C et al, J. Gen. Virol. 88 (2007) p 1062. Additionally, PrP^{Sc} was found to co-localise with heparan sulfate, and this heparan sulfate had an unusually under-sulfated motif. Attempts to synthesize the active saccharide epitope of this heparan sulfate have been made in order to investigate its possible role in scrapie pathogenesis. Both the GAG chains and the protein core of HSPGs were immunolocalised to the prion protein amyloid plaques in CJD and scrapie. Like PrP, GAGs have been demonstrated to effect changes in cancer too. Metabolism of GAGs was shown to be disrupted in prion diseases. Thus GAGs are secreted in the urine of prion-infected animals and humans as well as in the urine of mice ablated for the PrP gene. Polysulfated polysaccharides stimulate endocytosis of PrP^{c} and alter cellular localization of PrP^{c} precursor. Binding, of PrP with heparan-like molecules was shown to be specific and HS increased the concentration of PrP in neuroblastoma cells. Significant changes in transcription patterns for HSPG synthesis in prion infected cells were seen, suggesting a link between sulfation and PrP^{Sc}.

Prion proteins with pathogenic mutations have also been reported to show enhanced binding to glycosaminoglycans (Yin S et al, Proc. Nat. Acad. Sci. 104 (2007) p 7546), and sulfated polysaccharides have been found to both inhibit and stimulate PrP^{Sc} formation (Kocisko D A et al, Antimicrob. Agents Chemother. 50 (2006) p 1034). Evidence has now accumulated to support the theory that there is a conformational change in a secondary PrP upon dimerisation through the template of the first mis-folded PrP.

In relation to amyloidosis, the build up of amyloid plaques or fibrils are thought to be caused by inefficient HSPG turnover by the PrP system, such that a disease-causing GAG/HSPG binding to amyloid protein causes a longer biological half life of the protein. This in turn leads to accumulation of excess protein which is precipitated out of solution in plaque form.

### Prion Like Proteins

The prion-like Doppel protein (Dpl, Behrens, A. et al, EMBO J. 21 (2002) p 3652) has many biochemical and structural properties in common with the cellular prion protein (PrP^{c}), and the shadoo protein (Premzl, M. et al, Gene 314 (2003) p 89) is also a protein with significant similarity to PrP. Multiplicity (but not, we suggest, redundancy) appears to exist in the genome for prion expression in the sense that these homologues to prion protein exist. Dpl was found to be permanently expressed in the Sertoli cells but at different levels according to species (Serres, C, et al, Biol. Reprod. 74 (2006) p 816.). Dpl has been shown to regulate male fertility by controlling aspects of male gametogenesis (Behrens, A., et al, EMBO J. 21 (2002) p 3652), and to be up-regulated in some tumours in a related way to malignancy level (Comincini, S., et al, Anticancer Res. 24 (2004) p 1507). It is possible that these homologues are better designed for their specific PrP-like role in the tissues where these proteins are over-expressed.

### 2. General Information

As used herein the term “derived from” shall be taken to indicate that a specified integer may be obtained from a particular source albeit not necessarily directly from that source.

Unless the context requires otherwise or specifically stated to the contrary, integers, steps, or elements of the invention recited herein as singular integers, steps or elements clearly encompass both singular and plural forms of the recited integers, steps or elements.

Throughout this specification, unless the context requires otherwise, the word “comprise”, or variations such as “comprises” or “comprising”, will be understood to imply the inclusion of a stated step or element or integer or group of steps or elements or integers but not the exclusion of any other step or element or integer or group of elements or integers.

Unless specifically stated otherwise, each feature described herein with regard to a specific embodiment of the invention, shall be taken to apply mutatis mutandis to each and every other embodiment of the invention.

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations or any two or more of said steps or features.

The present invention is not to be limited in scope by the specific examples described herein. Functionally-equivalent products, compositions and methods are clearly within the scope of the invention, as described herein.

All the references cited in this application are specifically incorporated by reference herein.

The present invention is performed without undue experimentation using, unless otherwise indicated, conventional techniques of molecular biology, microbiology, virology, recombinant DNA technology, peptide synthesis in solution, solid phase peptide synthesis, and immunology. Such procedures are described, for example, in the following texts that are incorporated by reference:
Sambrook, Fritsch & Maniatis, Molecular Cloning: A Laboratory Manual, Cold Spring Harbor Laboratories, New York, Second Edition (1989), whole of VoIs I, II, and III; DNA Cloning: A Practical Approach, Vols. I and II (D. N. Glover, ed., 1985), IRL Press, Oxford, whole of text;
Oligonucleotide Synthesis: A Practical Approach (M. J. Gait, ed., 1984) IRL Press, Oxford, whole of text, and particularly the papers therein by Gait, pp 1-22; Atkinson et al, pp 35-81; Sproat et al, pp 83-115; and Wu et al, pp 135-151;
Nucleic Acid Hybridization: A Practical Approach (B. D. Hames & S. J. Higgins, eds., 1985) IRL Press, Oxford, whole of text;
Immobilized Cells and Enzymes: A Practical Approach (1986) IRL Press, Oxford, whole of text; Perbal, B., A Practical Guide to Molecular Cloning (1984);
Methods In Enzymology (S. Colowick and N. Kaplan, eds., Academic Press, Inc.), whole of series;
J. F. Ramalho Ortigão, “The Chemistry of Peptide Synthesis” In: Knowledge database of Access to Virtual Laboratory website (Interactiva, Germany);
Iozzo, Proteoglycan protocols (2001), Humana Press;
Leteux, C, et al, J. Biol. Chem. 276 (2001) p 12539;
Toshihiko et al., Trends in Glycoscience and Clycotechnology, 15 (2003) p 29.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this application.

### SUMMARY OF THE INVENTION

The present invention relates to the treatment of prion protein-related diseases, such as for example cancers (in particular tumours), amyloid diseases (eg amyloidosis and Alzheimer's disease), inflammatory conditions, transplant technology and associated ischemia.

It will be understood that as used herein the term “prion protein-related disease” encompasses a disease that is related to prion proteins or prion like proteins such as for example Shadoo and/or Doppel proteins. The present invention does not relate to what are thought to be “classical” prion diseases that are thought to be caused by prion infection, such as TSEs, in particular it is not intended to include within its scope prion diseases such as Creutzfeldt-Jakob disease, Gerstmann-Straussler-Scheinker syndrome, fatal familial insomnia or kuru. Rather the present invention relates to related diseases that are not necessarily caused by prion protein misfolding alone.

Most surprisingly, the inventors have found in their work leading to the present invention that prion proteins bind to and interact with specific glycosaminoglycans (GAGs) and/or heparan sulfate proteoglycans (HSPGs) to cause or assist in the process of disease. Those GAGs and HSPGs that bind to prion proteins and prion-like proteins and which are identified as being upregulated in certain disease states such as in cancer and/or which otherwise cause or assist in the process of disease are referred to herein as “disease-related”.

According to the present invention, treatment of prion protein related diseases, and in particular cancer tumours, is effected by providing to a subject in need thereof a therapeutically effective amount of an agent capable of modulating binding of a prion protein and/or a prion-like protein to a disease related GAG and/or HSPG.

The inventors have shown that antibodies or fragments of antibodies to these disease related GAGs, HSPGs, prion proteins and/or prion-like proteins can be used to treat or ameliorate a prion protein related disease or condition or complication thereof in a subject. The inventors have also shown that selected competitive binding glycosaminoglycans (GAGs), synthetic polysulfated polysaccharides, heparin sulphate proteoglycans (HSPGs), and pharmaceutical compositions comprising these, can be used to inhibit the binding and/or interaction of prion proteins and/or prion-like proteins to disease related GAGs and/or HSPGs and can be therapeutically useful to treat or ameliorate disease, including for example to prevent or slow the growth of a tumour in a subject. These competitive binding agents are particularly useful for treating cancers and amyloid diseases.

In summary then, the therapy provided in the present invention is understood to encompass the modulation of binding or disruption of binding of a prion protein and/or prion-like protein to a disease related GAG and/or HSPG. This modulation is also understood to include the disruption of a complex comprising a prion protein or prion like protein with a disease related GAG and/or HSPG, or preventing or decreasing formation thereof.

Accordingly, in a first aspect of the present invention there is provided a method for the treatment of a prion protein-related disease which comprises administering to a subject in need thereof an effective amount of an agent capable of modulating binding of a prion protein or a prion-like protein to a disease related GAG and/or HSPG.

In a preferred embodiment the prion protein is PrP^{c}. In another embodiment the prion like protein is a Shadoo or Doppel protein.

In one embodiment the agent is an antibody or antibody fragment capable of modulating binding of a prion protein or a prion-like protein to a disease related GAG and/or HSPG.

In one preferred embodiment, the agent is an anti-HSPG or anti-GAG antibody or fragment thereof.

In another embodiment, the agent is an anti-prion protein antibody (that is, an anti-PrP antibody) or an anti-prion-like protein such as for example an anti-Dpl or anti-Shadoo protein antibody, or fragment thereof.

Preferably the antibody is capable of specifically binding to PrP, Dpi or shadoo proteins and/or to a disease related HSPG or GAG.

Preferably, the antibody according to any of these embodiments is a monoclonal antibody, or fragment thereof. It is to be understood that fragments of antibodies according to the invention are immunologically effective portions.

In one embodiment, the monoclonal antibody is an antibody to PrP such as for example 1E5/G6, 3B8/D5, 3F4, 4H7, 5121, 5B2, 6G3, 7B6, 7D9, 8B4, C-20, FL-253, M-20, WD3C7 (Santa Cruz), 3C10, 5G12 (Jena), 6H4, 34C9 (Prionics), 3C8, 8H4 (Alicon), BAR221, BAR236, SAF83, SAF32, SAF53, SAF54 (SPIBio).

In another embodiment, the monoclonal antibody is an antibody to Dpl such as for example; 10005517 (Cayman).

In another embodiment the monoclonal antibody is an antibody to HSPG or GAG such as for example; 10E4 (Seikagaku), B-A38, BC/B-B4, CSI 001-74, CSI 001-76, SPM255 (Abeam), 1C9 (Abnova), 297716, 307801, 300712, 300736 (R&D Systems), 1G12 (Santa Cruz), A71, A74, A76 (AntibodyShop), A7L6 (Chemicon), 7B5 (Invitrogen).

In a preferred embodiment, the monoclonal antibody or fragment is humanised.

In another embodiment, the fragment is a Fab, Fab′ F(ab′)₂ fragment, or an Fᵥ of said monoclonal antibody.

In another embodiment the agent is an anti-inhibitory peptide antibody.

In still further features in the described preferred embodiments, an antigen recognition domain of the antibody or fragment thereof is encompassed by MAP amino acid sequences as follows:

SEQ ID NO: 1
KMMERVVEQMCITQYERESQ,
SEQ ID NO: 2
SAMSRPLIHFGSDYEDRYYRE,
SEQ ID NO: 3
TNMKHMAGAAAAGAWGGLG,
SEQ ID NO: 4
GWGQGGGTHSQWNKPSK,
and
SEQ ID NO: 5
RYPPQGGGGWGQPHGGG,.

In yet another embodiment, the present invention provides a hybridoma capable of producing a monoclonal antibody having specificity for a prion protein or to a disease related HSPG or GAG.

In one preferred embodiment, the antibody is administered with a cytotoxic agent. In one embodiment the cytotoxic agent is a chemotherapeutic agent. Examples of known cytotoxic agents include but are not limited to irinotecan, gemcitabine, doxorubicin, adriamycin, methotrexate, paclitaxel, cisplatin, oxaliplatin, 5-FU or vinorelbine.

In one embodiment the cytotoxic agent is conjugated to the antibody.

In an alternate embodiment of the invention, the modulating agent is selected from the group consisting of glycosaminoglycans (GAGs), synthetic polysulfated polysaccharides, and heparan sulphate proteoglycans (HSPGs).

Preferably the synthetic polysulfated polysaccharide, GAG or HSPG is one that competitively binds to a prion protein. Preferably the agent is a non-disease causing GAG or HSPG or synthetic polysulfated polysaccharide. In this way, amplification and/or signal transmission of the infectious or disease related GAG or HSPG can be competitively inhibited, by a non-disease causing agent.

In one embodiment a GAG modulating agent comprises four saccharides such as UA-GlcN-UA-GlcNAc.

In one embodiment the modulating agent is a heparan sulfate proteoglycan that is selected from the syndecan or glypican cell surface proteoglycans, or derived therefrom. In another embodiment the proteoglycan is a perlecan or serglycin or other heparan sulfate proteoglycan. In one preferred embodiment the modulating agent is derived from glypican-1, glypican-2, glypican-3, glypican-4, glypican-5, glypican-6, lumican, perlecan, syndecan-1, syndecan-2, syndecan-3, syndecan-4 or serglycin.

In one embodiment, the GAG modulating agent comprises a sequence that is substantially similar to an epitopic determinant of HSPG that is bound by antibody 10E4 and, preferably, which includes the sequence UA-GlcN-UA-GlcNAc.

By substantially similar is meant preferably 80% or greater homology, more preferably 90% or 95% or greater homology.

In another embodiment the modulating agent is a pentosan polysulfate such as xylopyranose polysulfate (XPS), which is a semi-synthetic derivative of beechwood.

In yet another embodiment a combination of modulating agents can be administered. In one embodiment an antibody is administered together with a cytotoxic agent. For example, an anti-PrP antibody can be administered together with a cytotoxic agent, an anti-PrP related protein antibody can be administered together with a cytotoxic agent, an anti-disease related GAG and/or HSPG antibody can be administered together with a cytotoxic agent.

In another embodiment an antibody is administered together with a GAG or HSPG. For example, an anti-PrP antibody can be administered together with a GAG and/or HSPG, an anti-PrP related protein antibody can be administered together with a GAG and/or HSPG, an anti-disease related GAG and/or HSPG antibody can be administered together with a GAG and/or HSPG.

In another embodiment a GAG and/or HSPG is administered together with a cytotoxic agent.

In another embodiment an antibody is administered together with a cytotoxic agent and a GAG and/or HSPG.

In yet another aspect, the invention provides for the use of an agent capable of modulating binding of a prion protein or a prion like protein to a disease related GAG or HSPG in the preparation of a medicament for treatment of a prion protein-related disease.

Furthermore, the invention provides a pharmaceutical composition comprising a GAG or HSPG or a variant or mimetic thereof (such as and not limited to synthetic polysulfated polysaccharides including pentosan polysulfate) or mixture thereof and/or an antibody (in some embodiments together with or conjugated to a cytotoxic agent), and a pharmaceutically acceptable carrier or diluent, for use according to the disclosures of the present invention.

The invention also includes a method of treating prion protein-related diseases, by administering a therapeutically effective amount of the pharmaceutical composition of the present invention to an animal in need thereof.

In another aspect the present invention provides a method of treatment of a subject in need thereof, said method comprising:

(i) identifying a subject suffering from prion protein related disease;
(ii) obtaining an amount of a composition according to any embodiment hereof; and
(iii) administering said composition to said subject.

In another example, the present invention also provides a method of treatment of a subject in need thereof, said method comprising:

(i) identifying a subject suffering from prion protein related disease; and
(ii) recommending administration of a composition according to any embodiment hereof.

In another example, the invention provides a method of treatment comprising administering or recommending a composition according to any embodiment hereof to a subject previously identified as suffering from prion protein related disease.

In another example, the invention provides a method of treatment comprising:

(i) identifying a subject suffering from prion protein related disease;
(ii) obtaining a composition according to any embodiment hereof;
(iii) formulating the composition at (ii) with a suitable carrier and/or excipient, e.g., for oral administration, topical administration, inhalation, injection or infusion, wherein said composition is in an amount sufficient to alleviate or prevent one or more symptoms or complications or the disease per se according to any embodiment hereof in a subject in need thereof and/or in an amount sufficient to inhibit, repress, delay or otherwise reduce binding or interaction of a prion protein or a prion like protein to a disease related GAG or HSPG; and
(iv) administering said formulation to said subject.

In yet another example, the present invention provides a method of treatment comprising:

(i) identifying a subject suffering from a prion protein related disease;
(ii) obtaining a composition according to any embodiment hereof;
(iii) formulating the composition at (ii) with a suitable carrier and/or excipient and
(iv) recommending a formulation at (iii).

In a particularly preferred example of the present invention, the method of treatment involves repeated administration, wherein each administration is timed so as to ensure a sufficiently high concentration of the bioactive compound or other composition of matter of the formulation in plasma of the subject in the treatment regimen.

According to another aspect of the invention there is provided a method of identifying or screening for a modulating agent candidate suitable for treating a prion protein-related disease. The method is effected by identifying a molecule capable of modulating binding of a prion to a specific GAG or HSPG, the molecule being the drug candidate.

In one embodiment, for example, the invention provides a method of identifying or screening for a candidate modulating agent which comprises use of an in vitro assay of the inhibition of the conversion of PrP^{sen} to PrP^{res}. The method can be effected by contacting the candidate agent with PrP^{sen} in the presence of PrP^{res} for a time and under suitable conditions for conversion of PrP^{sen} to PrP^{res} to occur, and determining if the conversion of PrP^{sen} to PrP^{res} has in fact occurred or is inhibited. Candidate compounds for a method of treatment according to the present invention inhibit conversion (see Example 3). The assay also provides a method of screening for variants, analogs and mimetics of the candidate modulating agents.

In a preferred embodiment the method comprises

(i) obtaining purified PrP^{res}
(ii) incubating purified PrP^{res} with PrP^{sen} in the presence or absence of a candidate agent; and
(iii) determining if PrP^{sen} has converted to PrP^{res},
wherein an absence of conversion to PrP^{res} indicates that the candidate agent is an effective modulating agent.

Preferably the PrP^{res} is purified.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

### BRIEF DESCRIPTION OF THE FIGURES

FIG. 1 is a graphical representation showing prion expression in near confluent cultures of breast cancer cell lines with varying invasiveness/malignancy.

FIG. 2 is a graphical representation showing PrP levels in colon cancer cell lines.

FIG. 3 is a graphical representation showing relative inhibitory effects of PrP antibody on HCT116 cells in MTT assay.

FIG. 4 is a graphical representation showing various antibody concentrations on growth of HCT116 cells in MTT assay.

FIG. 5 is a graphical representation showing IC50 ratio of IRI vs IRI/2A5 Ab.

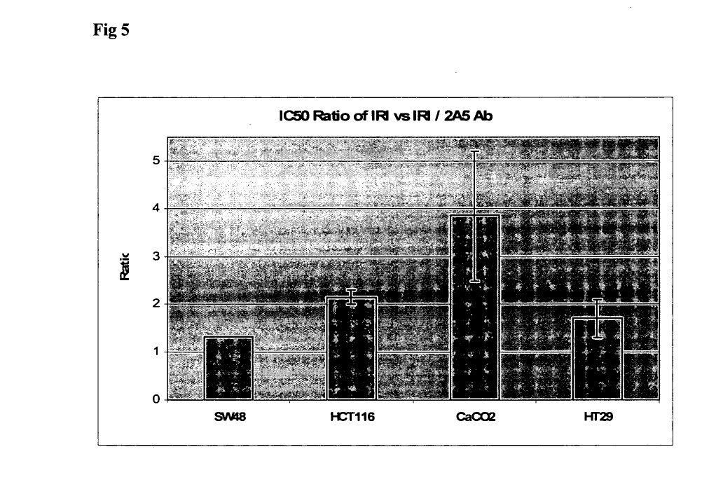

FIG. 6 is graphical representation showing antiproliferative response to HS antibody 10E4 in human colon cancer HCT116 cells.

FIG. 7 shows changes in HCT116 colon cancer cell morphology after 24 hours treatment with BAR221 anti-PrP antibody (right) compared to controls (left). Images are at 50× (A) and 200× (B) magnification.

FIG. 8 is a graphical representation of changes in protein expression upon treatment with BAR221 anti-PrP antibody as analysed by slot-blot. 10E4 HS expression increased whereas anti-apoptosis marker Bcl-2 decreased compared to beta-actin control.

FIG. 9 is a graphical representation of 10E4 HS expression in different breast cancer cell lines. Increased expression was correlated with decreased invasiveness.

FIG. 10 is a graphical representation of HCT116 tumour xenograft size in nude mice upon treatment with anti-PrP antibody.

FIG. 11 demonstrates a lower HCT116 xenograft tumour growth rate upon combination therapy with anti-PrP antibody compared with irinotecan alone.

FIG. 12 is a graphical representation of the effectiveness of pentosan polysulfate combination therapy compared to irinotecan alone in WiDr tumour xenografts.

FIG. 13 is a Kaplan-Meier plot showing increased survival of WiDr tumour xenograft-bearing mice receiving pentosan polysulfate combination therapy compared to mice receiving irinotecan alone.

### DETAILED DESCRIPTION OF THE INVENTION

### Abbreviations

### CJD Creutzfeldt-Jacob Disease

Dpl Doppel protein

### GAG Glycosaminoglycan

HS Heparan sulfate
HSPG Heparan sulfate proteoglycan

### PG Proteoglycan

PrP Prion protein
PrP^{c} Prion protein cellular form
PrP^{res} Prion protein resistant to proteinase K
PrP^{Sc} Prion protein scrapie form (disease form)
PrP^{sen} Prion protein sensitive to proteinase K

### PK Proteinase K

### TSE Transmissible Spongiform Encephalopathy

In work leading to the present invention, the inventors have surprisingly found that cellular prion protein (PrP^{c}), and prion-like proteins doppel protein (Dpl) and shadoo protein are involved in cell messaging systems which involve glycosaminoglycans (GAGs) and heparan sulfate proteoglycans (HSPGs).

The inventors postulated that one of the roles of prion proteins and prion-like proteins is in the transport, amplification and transmission of HSPG and/or GAGs.

As used herein the term “transport” refers to intra-cellular or inter-cellular movement of HSPG and/or GAGs. As used herein the term “amplification” refers to increased amount of the component such as for example GAG or HSPG. As used herein the term “transmission” refers to an infection or disease passed from a diseased cell or animal to a previously healthy cell or animal.

Referring to Example 4, the inventors propose that particular HS epitopes are amplified in a Protein Misfolding Cyclic Amplification (PMCA) procedure together with amplification of PrP^{Sc}.

The inventors confirmed that tumour cells (in this case colon cancer cells) over express prion protein (PrP^{Sc})(see Example 5) and the inventors surprisingly found that heparan sulfate and prion protein co-localise in cancer cells with both cell surface and endosomal distribution. Co-localisation was also evidenced during intercellular transport and cell division (see Example 6). This work suggests that the prion protein/GAG/HSPG machinery is interrelated and upregulated in cancer cells.

Most surprisingly, the inventors have shown that this mechanism can be inhibited as a treatment for cancers.

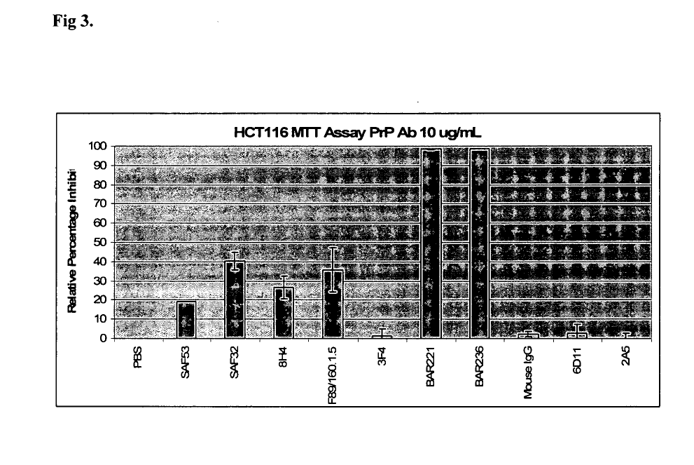

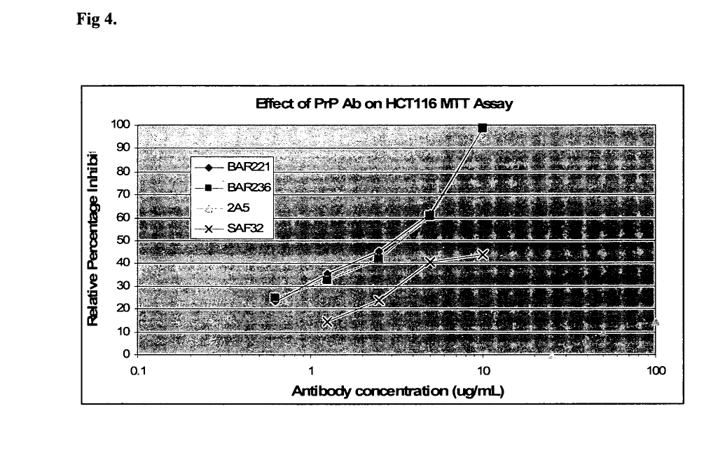

Referring to the Examples, in Examples 7 and 8 the inventors treated an animal having a tumour with anti-PrP antibody. The inventors compared tumour mass and cellular proliferation in animals treated with anti-PrP antibody with control animals not treated with anti-PrP antibody. The inventors found that treatment prevented or decreased tumour growth and/or cellular proliferation. The antibody used in these examples was 6D11. More preferably the antibody to be used is BAR221 or BAR226, or any other antibody that effectively inhibits growth of cancer cells such as for example HCT116 cells in a MTT assay (see FIGS. 3 and 4). Further the inventors showed in Example 9 that antibodies (in this case anti PrP antibodies) cause or increase apoptosis of cancer cells.

In Example 10 the inventors demonstrated that cancer cell proliferation and/or tumour mass growth was inhibited or suppressed or reversed by administration of a combination of anti-PrP antibody together with a cytotoxic agent (in this case irinotecan). According to the invention the effect of anti-PrP antibody was greater than the effect of the cytotoxin alone providing an enhanced treatment for a prion related disease such as cancer and in particular tumours.

In Example 11 the inventors demonstrated that HS antibody 10E4 prevents proliferation of cancer cells (as measured in MTT assay against human colon cancer HCT116 cells). According to the results there was a significant variance of the relative percentage of inhibition between control cells compared to cells treated with HS 10E4 antibodies.

Finally the inventors demonstrated that administration of a GAG, namely pentosan polysulfate in combination with an anti-PrP antibody and cytotoxic drug is even more effective to treat prevent reduce tumour mass or tumour cell proliferation.

Thus, in summary the inventors have found that cells that are attempting to change a phenotype of their environment or maintain strong abnormal phenotypic changes, such as cancer cells, are found to over express PrP, Dpl, and/or shadoo proteins as well as certain disease related GAGs and HSPGS compared to normal tissues and that tumour growth can be inhibited by the administration of an agent that interrupts the PrP, Dpl or shadoo protein amplification, transport or transmission systems.

The inventors propose that specific GAGs and/or HSPGs (herein referred to as “disease related GAGs and HSPGs”) which are isolated from cells or animals infected with a prion disease such as transmissible spongiform encephalopathies (TSEs), can be amplified with this prion protein cell messaging system and can also be used to infect normal cells or animals, to transmit or cause various disease pathologies in the previously uninfected cells or animals.

The invention proposes a method for amplifying specific GAG sequences or HSPG sequences using the native prion protein or prion like protein. As described in Examples 13 and 14 the method involves obtaining a HSPG to be amplified, contacting the HSPG with a brain homogenate or a fraction of brain homogenate or with the necessary enzymes/components of brain homogenate, for a time under suitable conditions for amplification to occur. Preferably the method involves incubation and/or sonication of the sample. Preferably the method includes isolating the amplified GAG or HSPG.

In one example, the inventors also postulate that a HSPG isolated from a neuroblastoma cell line infected with scrapie is capable of causing scrapie in an uninfected neuroblastoma culture. The treatment of the HSPG with heparinase could be used to show removal of infectivity in this model.

The unusual discovery by the inventors that prion protein is involved in the transport, amplification and/or transmission of GAG/HSPG signaling, has enabled the inhibition of this mechanism as a treatment for other related disorders such as amyloidosis and Alzheimer's disease and in transplant technology. With regard to amyloidosis, the build up of amyloid plaques or fibrils are thought to be caused by inefficient HSPG turnover by the PrP system, such that a disease-causing GAG/HSPG binding to amyloid protein causes a longer biological half life of the protein. This in turn leads to accumulation of excess protein which is precipitated out of solution in plaque form.

Expression of PrP in Alzheimer's disease has been noted: McNeill, A., MUM 8 (2004) p 7-14, and similarly, in acute signaling situations such as in organ transplant, upregulation of GAG/HSPG signaling rapidly takes place. This signaling, leading to ischemia, can be minimized by inhibition of the PrP mechanism, by methods according to the present invention.

Thus according to the present invention there is provided a method for the treatment of a prion protein-related disease including for example cancer, amyloid diseases and inflammation which comprises administering to a subject in need thereof an effective amount of an agent capable of modulating binding of a PrP, shadoo or doppel protein to a disease related GAG or HSPG.

A modulating agent can be, for example, a monoclonal or polyclonal antibody or an antibody fragment capable of binding a PrP, shadoo or doppel protein, or a disease related GAG or HSPG. Preferably, the antibody specifically binds at least one epitope of PrP, shadoo or doppel protein or specifically identified GAG or HSPG. In alternate aspects, such as for the specific treatment of cancers and amyloid diseases, the modulating agent can also be a GAG, HSPG or synthetic polysulfated polysaccharide preferably that specifically and competitively binds to a prion, doppel or shadoo protein.

The term “specific binding” refers to an agent capable of binding substantially only to a defined target.

In preferred embodiments of the invention the modulating agent comprises a combination of an antibody and/or GAG and/or HSPG and/or synthetic polysulfated polysaccharide and/or cytotoxic agent.

### Prion Proteins and Prion-Related Proteins

PrP is an animal protein that is the translation product of the PrP gene, wherein the protein consists of 253 amino acids in humans (as disclosed in Kretzschmar et al., DNA 5:315-324, 1986; Pucket et al., Am. J. Hum. Genet. 49:320-329, 1991), 254 amino acids in hamsters and mice, 264 amino acids in cows, and 256 amino acids in sheep. The amino acid sequences of all of these proteins are known, and are disclosed in U.S. Pat. No. 5,565,186, as well as in Locht, C. et al., Proc. Natl. Acad. Sci. USA 83:6372-6376, 1986; Kretzschmar, H. A. et al., DNA 5:315-324, 1986; Yoshimoto, J. et al., Virus Genes 6:343-356, 1992; and Goldmann, W. et al. Proc. Natl. Acad. Sci. USA 87:2476-2480, 1990. The PrP protein includes a native PrPsen (PrP^{c}) isoform which is degraded by proteinase K, and a pathological PrP^{res} (PrP^{Sc}) form which is partially resistant to proteinase K, and which induces a conformational change in PrP^{sen} to form characteristic amyloid deposits of the type seen in the spongiform encephalopathies.

As used herein the term “PrP” refers generically to proteins from animals, and includes specific human, hamster, murine, sheep, bovine or avian forms of the PrP. Inhibitory PrP peptides and cDNAs are orthologs of the disclosed murine and human PrP sequences and are thus structurally related by the possession of similar amino acid and nucleic acid structures. The region from positions 119-136 of the PrP is identical in humans (P113-136), mouse (P112-119), mink, rat, sheep (P116-123), cow (P124-131), Chinese hamster and Armenian hamster. Sequences are substantially homologous across even longer regions in different species. For example mouse and human sequences are identical across a window of 113-141, except for an Ile for Met substitution at position 138 in the aligned mouse sequence. The hamster and human sequences are identical across a window of P113-141, except for a Ile for Met substitution at each of positions 138 and 139 of the human sequence. The mouse and hamster sequences are identical across a comparison window of P109-141, except for a Met to Leu substitution at each of P109 and P112, and a Met to Ile substitution in the mouse sequence.

Shadoo protein and doppel protein are homologues of the prion protein. The shadoo protein sequence is provided in Lampo, E., et al, BMC Genomics 8 (2007) page 138 and a recent review on the doppel protein is Comincini, S., et al, Central European Journal of Biology 1 (2006) page 494.

### Antibodies and Epitopes

Examples of antibodies suitable for use as modulating agents in the present invention include for example monoclonal antibodies to PrP such as for example; BAR221, BAR236, SAF83, SAF32, SAF53, SAF54 (SPIBio), 1E5/G6, 3B8/D5, 3F4, 4H7, 5121, 5B2, 6G3, 7B6, 7D9, 8B4, C-20, FL-253, M-20, WD3C7 (Santa Cruz), 3C10, 5G12 (Jena), 6H4, 34C9 (Prionics), 3C8, 8H4 (Alicon). Antibodies to Dpi include, for example, 10005517 (Cayman). Antibodies to HSPG/GAG include for example, 10E4 (Seikagaku), B-A38, BC/B-B4, CSI 001-74, CSI 001-76, SPM255 (Abeam), 1C9 (Abnova), 297716, 307801, 300712, 300736 (R&D Systems), 1G12 (Santa Cruz), A71, A74, A76 (AntibodyShop), A7L6 (Chemicon), 7B5 (Invitrogen).

Alternative names for Prion protein PrP antibody [8H4] (ab61409) include for example ASCR antibody, fatal familial insomnia antibody, CD230 antigen antibody, CJD antibody, Creutzfeld Jakob disease antibody, Gerstmann-Strausler-Scheinker syndrome antibody, GSS antibody, Major prion protein antibody, MGC26679 antibody, Prion related protein antibody, PRIP antibody, Prni antibody, Prnp antibody, PrP antibody, PrP27-30 antibody, PrP33-35C antibody, PrPC antibody, PrPSc antibody, and Sine antibody.

According to the present invention, in one embodiment, an antibody of the invention specifically binds to an antigen encompassed by amino acid sequences (PrP human):

SEQ ID NO: 1
KMMERWEQMCITQYERESQ,
SEQ ID NO: 2
SAMSRPLIHFGSDYEDRYYRE,
SEQ ID NO: 3
TNMKHMAGAAAAGAWGGLG,
SEQ ID NO: 4
GWGQGGGTHSQWNKPSK,
and
SEQ ID NO: 5
RYPPQGGGGWGQPHGGG,.

As used herein, the term “epitope” refers to any antigenic determinant on an antigen to which the paratope of an antibody binds.

Epitopic determinants are composed of chemically active surface groupings of molecules such as amino acids or carbohydrate side chains and usually have specific three-dimensional structural characteristics, as well as specific charge characteristics. Preferred epitopes for the present invention include those comprising the above sequences, for example.

The term “antibody” as used herein includes any specific binding substance having a binding domain with the required specificity and/or affinity for prion protein or a prion-like protein (or fragment or epitope thereof) or disease related GAG or HSPG. The term antibody includes intact molecules as well as functional fragments thereof, such as Fab, F(ab′)2 and Fv. These functional antibody fragments are described as follows:
1. Fab, the fragment which contains a monovalent antigen-binding fragment of an antibody molecule, can be produced by digestion of whole antibody with the enzyme papain to yield an intact light chain and a portion of one heavy chain;
2. Fab′, the fragment of an antibody molecule that can be obtained by treating whole antibody with pepsin, following by reduction, to yield an intact light chain and a portion of the heavy chain; two Fab′ fragments are obtained per antibody molecule;
3. (Fab′)₂, the fragment of the antibody that can be obtained by treating whole antibody with the enzyme pepsin without subsequent reduction; F(ab′)2 is a dimer of two Fab′ fragments held together by two disulfide bonds;
4. Fv, defined as a genetically engineered fragment containing the variable region of the light chain and the variable region of the heavy chain expressed as two chains; and
5. Single chain antibody (“SCA”), a genetically engineered molecule containing the variable region of the light chain and the variable region of the heavy chain, linked by a suitable polypeptide linker as a genetically fused single chain molecule.

The term antibody includes conjugates of antibodies and fragments. Further the term antibody shall also be taken to include a cell expressing an antibody or part thereof.

Methods of producing polyclonal and monoclonal antibodies as well as fragments thereof are well known in the art (see for example, Harlow and Lane, Antibodies: A Laboratory Manual, Cold Spring Harbor Laboratory, New York, 1988, incorporated herein by reference).

For example an antibody, preferably a monoclonal antibody, is produced by immunizing an animal (e.g., a mouse) with the relevant immunogen. Optionally, the immunogen is injected in the presence of an adjuvant, such as, for example Freund's complete or incomplete adjuvant, lysolecithin and/or dinitrophenol to enhance the immune response to the immunogen. The immunogen may also be linked to a carrier protein, such as, for example, BSA. Spleen cells are then obtained from the immunized animal. The spleen cells are then immortalized by, for example, fusion with a myeloma cell fusion partner, preferably one that is syngenic with the immunized animal. A variety of fusion techniques may be employed, for example, the spleen cells and myeloma cells may be combined with a nonionic detergent or electrofused and then grown in a selective medium that supports the growth of hybrid cells, but not myeloma cells. A preferred selection technique uses HAT (hypoxanthine, aminopterin, thymidine) selection. After a sufficient time, usually about 1 to 2 weeks, colonies of hybrids are observed. Single colonies are selected and growth media in which the cells have been grown is tested for the presence of binding activity against the polypeptide (immunogen). Hybridomas having high reactivity and specificity are preferred.

Monoclonal antibodies are isolated from the supernatants of growing hybridoma colonies using methods such as, for example, affinity purification using the immunogen used to immunize the animal to isolate an antibody capable of binding thereto. In addition, various techniques may be employed to enhance the yield, such as injection of the hybridoma cell line into the peritoneal cavity of a suitable vertebrate host, such as a mouse. Monoclonal antibodies are then harvested from the ascites fluid or the blood of such an animal subject. Contaminants are removed from the antibodies by conventional techniques, such as chromatography, gel filtration, precipitation, and/or extraction.

Antibody fragments according to the present invention can be prepared for example by proteolytic hydrolysis of the antibody or by expression in E. coli or mammalian cells (e.g. Chinese hamster ovary cell culture or other protein expression systems) of DNA encoding the fragment. Antibody fragments can be obtained by pepsin or papain digestion of whole antibodies by conventional methods. For example, antibody fragments can be produced by enzymatic cleavage of antibodies with pepsin to provide a 5S fragment denoted F(ab′)2. This fragment can be further cleaved using a thiol reducing agent, and optionally a blocking group for the sulfhydryl groups resulting from cleavage of disulfide linkages, to product 3.5S Fab′ monovalent fragments. Alternatively, an enzymatic cleavage using pepsin produces two monovalent Fab′ fragments and an Fc fragment directly. These methods are described, for example, by Goldenberg, U.S. Pat. Nos. 4,036,945 and 4,331,647 and references contained therein, which patents are hereby incorporated by reference in their entirety. See also Porter, R. R. [Biochem. J. 73:119-126 (1959)]. Other methods of cleaving antibodies, such as separation of heavy chains to form monovalent light-heavy chain fragments, further cleavage of fragments, or other enzymatic, chemical, or genetic techniques may also be used, so long as the fragments bind to the antigen that is recognized by the intact antibody.

Fv fragments comprise an association of VH and VL chains. This association may be noncovalent, as described in Inbar et al., [Proc. Nat'l Acad. Sci. USA 69:2659-62 (1972)]. Alternatively, the variable chains can be linked by an intermolecular disulfide bond or cross-linked by chemicals such as glutaraldehyde. Preferably, the Fv fragments comprise VH and VL chains connected by a peptide linker. These single-chain antigen binding proteins (scFv) are prepared by constructing a structural gene comprising DNA sequences encoding the VH and VL domains connected by an oligonucleotide. The structural gene is inserted into an expression vector, which is subsequently introduced into a host cell such as E. coli.

The antibody utilized by the present invention is preferably an antibody fragment which is capable of being delivered to, or expressed in, mammalian cells.

Another form of an antibody fragment is a peptide coding for a single complementarity-determining region (CDR). CDR peptides (“minimal recognition units”) can be obtained by constructing genes encoding the CDR of an antibody of interest. Such genes are prepared, for example, by using the polymerase chain reaction to synthesize the variable region from RNA of antibody-producing cells. See for example, Larrick and Fry [Methods, 2:106-10 (1991)].

Humanized forms of non-human (e.g. murine) antibodies are chimeric molecules of immunoglobulins, immunoglobulin chains or fragments thereof (such as Fv, Fab, Fab¹, F(ab′)₂ or other antigen-binding subsequences of antibodies) which contain minimal sequence derived from non-human immunoglobulin. Humanized antibodies include human immunoglobulins (recipient antibody) in which residues form a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin [Jones et al., Nature, 321:522-525 (1986); Riechmann et al., Nature, 332:323-329 (1988); and Presta, Curr. Op. Struct. Biol., 2:593-596 (1992)].

Methods for humanizing non-human antibodies are well known in the art. Generally, a humanized antibody has one or more amino acid residues introduced into it from a source which is non-human. These non-human amino acid residues are often referred to as import residues, which are typically taken from an import variable domain. Humanization can be essentially performed following the method of Winter and co-workers [Jones et al., Nature, 321:522-525 (1986); Riechmann et al., Nature, 332:323-327 (1988); Verhoeyen et al., Science, 239:1534-1536 (1988)], by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

Human antibodies can also be produced using various techniques known in the art, including phage display libraries [Hoogenboom and Winter, J. MoI. Biol, 227:391 (1991); Marks et al., J. MoI. Biol, 222:581 (1991)]. The techniques of Cole et al., and Boerner et al., are also available for the preparation of human monoclonal antibodies (Cole et al., Monoclonal Antibodies and Cancer Therapy, Alan R. Liss, p. 77 (1985) and Boerner et al., J. Immunol, 147(1):86-95 (1991)]. Similarly, human antibodies can be made by introduction of human immunoglobulin loci into transgenic animals, e.g. mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016 and in the following scientific publications: Marks et al., Bio/Technology 10:779-783 (1992); Lonberg et al., Nature 368: 856-859 (1994); Morrison, Nature 368: 812-813 (1994); Fishwild et al., Nature Biotechnology 14, 845-851 (1996); Neuberger, Nature Biotechnology 14: 826 (1996); and Lonberg and Huszar, Intern. Rev. Immunol. 13, 65-93 (1995).

Methods for assessing efficacy of any amount of antibody or antibody fragment or conjugate for treating a prion protein disease in a subject or a complication associated with a prion protein disease will be apparent to the skilled artisan from the disclosure herein. For example, a composition comprising an amount of an antibody or antibody fragment or conjugate is administered to a population of subjects infected with a prion protein disease and the number of subjects in which severity of the disease of a symptom thereof or a complication thereof is reduced is determined. An amount of an antibody or antibody fragment or conjugate that reduces the severity of the disease or symptom or complication thereof in a significant proportion of the population is considered to be an amount of an antibody or antibody fragment or conjugate sufficient to treat a prion protein disease. For example an effective amount of antibody, fragment or conjugate reduces the severity of the disease or symptom or complications in at least about 50% of the population or at least about 60% of the population, or at least about 70% of the population, or at least about 80% of the population or at least 90% of the population or at least about 95% of the population.

### Cytotoxic Drugs

According to the present invention, a combination of antibody and cytotoxic drug provides an improved therapy for prion related diseases such as cancer and in particular tumours, compared to cytotoxic drugs alone. Cytotoxic drugs preferably include, but are not limited to irinotecan, gemcitabine, doxorubicin, adriamycin, methotrexate, paclitaxel, cisplatin, oxaliplatin, 5-FU or vinorelbine. It is to be understood that the cytotoxic drug should be suitable for use and compatible to be administered to a patient in need thereof.

The following is a list of cytotoxic drugs currently used, however this listing is not exhaustive.

| Drug | Trade name |
|---|---|
| Altretamine | Hexalen |
| Amsacrine | Amsidyl |
| L-Asparaginase | See Colaspase |
| Bleomycin | Blenoxane, Blenamax, Bleomycin |
| Busulfan | Myleran |
| Capecitabine | Xeloda |
| Carboplatin | |
| Carmustine | Bicnu |
| Chlorambucil | Leukeran |
| Cisplatin | Cisplatin |
| Cladribine | Leustatin, Litak |
| Colaspase | Leunase |
| Cyclophosphamide | Cycloblastin, Endoxan |
| Cytarabine | Cytarabine |
| Dacarbazine | Dacarbazine |
| Dactinomycin | Cosmegen |
| Daunorubicin | Daunorubicin |
| Daunorubicin liposomal | Dauno Xome |
| Docetaxel | Taxotere |
| Doxorubicin | Adriamycin, Doxorubicin |
| Doxorubicin liposomal | Caelyx |
| Epirubicin | Pharmorubicin, Epirubicin |
| Etoposide | Phosphate Etopophos |
| Etoposide | Etoposide, Vepesid |
| Fluorouracil | Efudix, Fluorouracil |
| Fludarabine | Fludara |
| Fotemustine | Muphoran |
| Ganciclovir | Cymevene |
| Gemcitabine | Gemzar |
| Hydroxyurea | Hydrea |
| Idarubicin | Zavedos |
| Ifosfamide | Holoxan |
| Irinotecan | Camptosar |
| Lomustine | Cee Nu |
| Melphalan | Alkeran |
| Mercaptopurine | Puri-nethol |
| Methotrexate | Ledertrexate, Methoblastin, Methotrexate |
| Mitoantrone | Novantrone, Mitozantrone, Onkotrone |
| Mitomycin-C | Mitomycin C |
| Nimustine | Nimustine |
| Oxaliplatin | Eloxatin |
| Paclitaxel | Anzatax, Paclitaxel Ebewe, Taxol |
| Pemetrexed | Alimta |
| Procarbazine | Natulan |
| Raltitrexed | Tomudex |
| Temozolomide | Temodal |
| Teniposide | Vumon |
| Thioguanine | Lanvis |
| Thiotepa | Thiotepa |
| Topotecan | Hycamtin |
| Vinblastine | Velbe, Vinblastine |
| Vincristine | Oncovin, Vincristine |
| Vindesine | Eldisine |
| Vinorelbine | Navelbine, Vinorelbine |

### Proteoglycans and Polysaccharides

### Disease Related GAGs/HSPGs

Disease related GAGs/HSPGs can be identified for example by analysis of the sugar sequence and or disaccharide composition of tumour and TSE tissue. This information is then compared to the sugar sequence of GAGs/HSPGs from normal tissue. GAGs/HSPGs that have a different from normal sugar composition/sequence are identified as being disease related. Differences may occur in the sites of sulfation, degree of sulfation or sugar sequence. Disaccharide composition may be accomplished as per Ha et al. Carbohydrate Res., 340 (2005) p 411-416, Lyon et al., J. Biol. Chem., 269 (1994) p 11208-11215 or Parthasarathy et al., J. Biol. Chem. 273 (1998) p 21111-21114. Sequencing may be accomplished as per Turnbull et al., Proc. Natl. Acad. Sci. USA, 96 (1999) p 2698-2703 or Vives et al., Biochem. J., 339 (1999) p 767-773.

### As Modulating Agents

In certain aspects of the invention, including for example, the treatment of cancers and amyloid diseases, modulating agents can be proteoglycans and polysaccharides such as HSPGs, GAGs and polysulfated polysaccharides. These can be natural or synthetic modulating agents. Methods for making the same are described in the examples and are well known as described in Iozzo, Proteoglycan protocols (2001), Humana Press, Leteux, C, et al, J. Biol. Chem. 216 (2001) p 12539, Toshihiko et al., Trends in Glycoscience and Glycotechnology, 15 (2003) p 29.

Preferably, the GAG or HSPG agents of the invention, specifically inhibit cell free conversion of PrP^{sen} to PrP^{res}, in an assay of the type disclosed in Example 3, with an IC₅₀ of less than about 1000 μM, for example less than about 600 μM, 550 μM, 200 μM, 120 [mu]M, or even 100 μM. Although the assay of Example 3 uses hamster PrP to determine inhibition of the conversion reaction (conversion of PrP^{sen} to PrP^{res}), human or other PrP may be substituted in the assay, particularly in instances where it is desired to test variants that are to be used in different species. For example, inhibition of the conversion reaction for human PrP may be tested by substituting human PrP for hamster PrP in the assay of Example 3.

In a preferred embodiment the competitive binding modulating agent comprises a short GAG (natural or synthetic), such as a GAG comprising at least 4-8 saccharides, more preferably 9, 10, 12, 14 or 15 saccharides. Such saccharides may be prepared from, for example, heparan sulfate depolymerised using heparin lyase III. Heparan sulfate with low sulfate content (4-9%) is preferred, with 10-90% depolymerisation.

In one embodiment a GAG modulating agent comprises four saccharides such as UA-GlcN-UA-GlcNAc.

Variant GAG/HSPG modulating agents are HSPG (such as glypican-1, glypican-2, glypican-3, glypican-4, glypican-5, glypican-6, lumican, perlecan, syndecan-1, syndecan-2, syndecan-3, syndecan-4 or serglycin), GAGs (such as GAGs and GAG fragments derived from glypican-1, glypican-2, glypican-3, glypican-4, glypican-5, glypican-6, lumican, perlecan, syndecan-1, syndecan-2, syndecan-3, syndecan-4 or serglycin), synthetic GAGs and polysulfated polysaccharides (such as dextran sulfate and pentosan polysulfate). Preferably all modulating agents retain the desired inhibitory activity, as readily measured by the cell free assay of inhibitory activity disclosed in the present specification.

The syndecan family contains four members (syndecan-1/syndecan, syndecan-2/fibroglycan, syndecan-3/N-syndecan, syndecan-4/ryudocan (amphyglycan)), which are transmembrane heparan sulfate proteoglycans (HSPGs)(1,2). These HSPGs exhibit cell type-specific distribution with vascular endothelial cells expressing syndecan-1, -2, and -4, and predominant targeting to basolateral surfaces. The syndecan family members are type I integral membrane proteins with homologous transmembrane and cytoplasmic domains. The combined transmembrane/cytoplasmic domains contain four well-conserved tyrosine residues, which might serve important roles for biological function.

The cytoplasmic tail of syndecan-1 interacts with intracellular microfilaments, and that of syndecan-4 with focal adhesion molecules. Syndecan-1 has both heparan sulfate and chondroitin sulfate GAG (glycosaminoglycan) chains with a tissue-specific structural polymorphism due to distinct post translational modifications. Syndecan-1 has also been purified as an anticoagulant HSPG from endothelial cells or as a bFGF receptor molecule in golden hamsters. Thus, this polymorphism of syndecan-1 likely reflects distinct HSPG functions.

The glypicans, another cell surface HSPG family, is composed of six members (glypican-1/glypican, glypican-2/cerebroglycan, glypican-3, glypican-4/K-glypican, glypican-5 and glypican-6). Glypican family members possess an extracellular region with GAG attachment sites, 14 invariant cysteine residues, which stabilize a highly compact tertiary structure, and a COOH-terminal GPI (glycosylphosphatidylinositol) anchor. Glypican family members are selectively expressed on different cell types with only glypican-1 present on vascular endothelial cells. These HSPGs are mainly targeted to apical surfaces, and this process is partially dependent upon the extent of glycanation. It is also suggested that glypican plays an important role in regulating the biological activity of fibroblast growth factors via HS GAG chains like syndecan.

Pentosan polysulfates (PPS) and, more particularly, xylopyranose polysulfates are available as an alkali metal salt or alkaline earth metal salt, for example, comprising calcium or sodium salt, or transition metals such as copper and zinc and noble metals such as platinum. Accordingly, the particular complexing ions may be selected from the group consisting of the alkali metals, e.g. Na+ and K+, alkaline earth metals, e.g. Ca2+, Zn2+, Mg2+, Ba2+, as well as Ag+, Pb2+, Cu2+, Au2+, Pd2+, Pd4+, Pd4+, Pd2+, trivalent metal ions, and quaternary ammonium compound complexes. Examples of the latter compound are pyridinium chloride, tetraalkyl ammonium chloride, choline chloride, cetylpyridinium chloride, N-cetyl-N,N,N-trialkylammonium chloride or their derivatives. The most preferred of these are the divalent alkaline earth metals, preferably calcium, and magnesium and most preferable is the calcium complex, with on average one methylglucuronic acid every tenth xylose residue. Preparation of the polysulfate polysaccharide-metal complexes is described in detail in U.S. Pat. No. 5,668,116, the entire disclosure of which is incorporated herein by reference.

Other polysulfated polysaccharides included within the scope of the invention are, for example, polysulfated dextran and derivatives thereof, polysulfated cyclodextrin, sulfated heparin, sulfated mannose and mannose derivatives, xylan, polysulfated chondroitin, dermatan and hyaluronic acid. Further examples are polysulfated polysaccharide derivatives of homopolysaccharides or heteropolysaccharides which can be linear or branched. As described above, complexes are also formed between these polysulfated polysaccharides and multivalent metal ions, Ag+ and Au+, and quaternary ammonium compound complexes.

The sugars may come from but are not limited to pentoses or hexoses such as galactose, mannose, glucose, rhamnose, fructose, sorbose, xylose, D-arabinose, ribose, L-arabinose, glucuronic acid and their derivatives.

The term oversulfated refers to the compound having a sulfate group attached to all oxygen sites that are available for sulfation. For example, PPS contains approximately two sulfate groups per carbohydrate monomer. Due to uronic acid side groups on PPS, the degree of sulfation on PPS is approximately 1.8.

Sulphation of polysaccharides is described in detail in U.S. Pat. No. 5,668,116, the entire disclosure of which is incorporated herein by reference.

### Therapeutics

The modulating agents of the present invention can be used in the treatments of the invention per se or as part of a pharmaceutical composition.

As used herein the term “treating” or refers to reversing, alleviating, slowing, inhibiting the progress of, or preventing the progress of the disease, disorder or condition to which such term applies, or one or more symptoms of such disorder or condition or a complication derived from the disease or condition. The term “treatment” or “therapy” refers to the act of treating the subject.

As used herein the phrase “a subject in need thereof refers to a mammal, preferably a human subject, having a prion protein-related disease or is at risk of developing a prion protein-related disease (i.e., predisposed).

As used herein, the terms “prevention” and “treatment” shall not be taken to require an absolute i.e., 100% abrogation of prion protein disease or related condition, or an absolute i.e., 100% prevention of the growth of a tumour in a subject having risk factors thereof, and it is sufficient that there is a significant reduction in the adverse symptoms using the method of the present invention compared to the absence of prophylaxis or therapy in accordance with the present invention.

Similarly, the term “alleviating” or “alleviate” as used throughout this specification shall not be taken to require abrogation of prion protein disease or related condition in a subject that is more than a significant effect compared to the absence of treatment in accordance with the present invention.

Similarly, the terms “inhibit”, “enhance”, “repress”, “delay”, “enhance”, “induce”, “activate” and “promote” as used throughout this specification shall not be taken to require any particular quantitative change, merely a modified level and/or activity and/or expression, or modified timing thereof, that is significant compared to the absence of treatment in accordance with the present invention.

As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism.

Hereinafter, the phrases “physiologically acceptable carrier” and “pharmaceutically acceptable carrier” which may be interchangeably used refer to a carrier or a diluent that does not cause significant irritation to an organism and does not abrogate the biological activity and properties of the administered compound. An adjuvant is included under these phrases.

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

As used herein the terms “suitable carrier” or “excipient shall be taken to mean a compound or mixture that is suitable for use in a composition that is to be administered to a subject. For example a suitable carrier or excipient for use in the invention for injection into a subject will generally not cause an adverse reaction in the subject.

Techniques for formulation and administration of drugs may be found in “Remington's Pharmaceutical Sciences,” Mack Publishing Co., Easton, Pa., latest edition, which is incorporated herein by reference.

Suitable routes of administration may, for example, include oral, rectal, transmucosal, especially transnasal, intestinal or parenteral delivery, including intramuscular, subcutaneous and intramedullary administration as well as intrathecal, direct intraventricular, intravenous, intraperitoneal, intranasal, or intraocular administration. This list is not meant to be exhaustive.

Alternately, one may administer the pharmaceutical composition in a local rather than systemic manner, for example, via injection of the pharmaceutical composition directly into a tumour tissue region of a patient. Deposition, implantation, controlled release and any other suitable method of administration are considered to be incorporated here.

Pharmaceutical compositions of the present invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with the present invention thus may be formulated in a conventional manner using one or more physiologically acceptable carriers comprising excipients and auxiliaries, which facilitate processing of the active ingredients into preparations which can be used pharmaceutically. Proper formulation is dependent upon the route of administration chosen.

For injection, the active ingredients of the pharmaceutical composition may be formulated in aqueous solutions, preferably in physiologically compatible buffers such as Hank's solution, Ringer's solution, or physiological salt buffer. One route of administration which is suited for the pharmaceutical compositions of the present invention is sub-periosteal injection, as described in U.S. Pat. No. 6,525,030 to Erikkson. For transmucosal administration, penetrants appropriate to the barrier to be permeated are used in the formulation. Such penetrants are generally known in the art.

For oral administration, the pharmaceutical composition can be formulated readily by combining the active compounds with pharmaceutically acceptable carriers well known in the art. Such carriers enable the pharmaceutical composition to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatine, gum tragacanth, methyl cellulose, hydroxypropylmethyl-cellulose, sodium carboxymethylcellulose; and/or physiologically acceptable polymers such as polyvinyl pyrrolidone (PVP). If desired, disintegrating agents may be added, such as cross-linked polyvinyl pyrrolidone, agar, or alginic acid or a salt thereof such as sodium alginate. As used herein, the term “oral administration” includes administration of the pharmaceutical compound to any oral surface, including the tongue, gums, palate, or other buccal surfaces. Addition methods of oral administration include provision of the pharmaceutical composition in a mist, spray or suspension compatible with tissues of the oral surface.

Dragee cores are provided with suitable coatings. For this purpose, concentrated sugar solutions may be used which may optionally contain gum Arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions which can be used orally include push-fit capsules made of gelatine, as well as soft, sealed capsules made of gelatine and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in a conventional manner.

For administration by nasal inhalation, the active ingredients for use according to the present invention are conveniently delivered in the form of an aerosol spray presentation from a pressurized pack or a nebuliser with the use of an aerosol spray presentation from a pressurized pack or a nebuliser with the use of a suitable propellant, e.g., dichlorodifluoromethane, trichlorofluoromethane, dichlorotetrafluoroethane or carbon dioxide. In the case of a pressurized aerosol, the dosage unit may be determined by providing a valve to deliver a metered amount. Capsules and cartridges of, e.g., gelatine for use in a dispenser may be formulated containing a powder mix of the compound and a suitable powder base such as lactose or starch.

The pharmaceutical composition described herein may be formulated for parenteral administration, e.g., by bolus injection or continuous infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oil or water based injection suspensions. Suitable lipophilic solvents or vehicles include fatty oils such as sesame oil, or synthetic fatty acid esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions.

Alternatively, the active ingredient may be in powder form for constitution with a suitable vehicle, e.g., sterile pyrogen-free water based solution, before use. The pharmaceutical composition of the present invention may also be formulated in rectal compositions such as suppositories or retention enemas, using, e.g., conventional suppository bases such as cocoa butter or other glycerides.

Pharmaceutical compositions suitable for use in context of the present invention include compositions wherein the active ingredients are contained in an amount effective to achieve the intended purpose. More specifically, a therapeutically effective amount means an amount of active ingredients (e.g. PrP antibody) effective to prevent, alleviate or ameliorate symptoms of a disorder (e.g., mammary tumor progression) or prolong the survival of the subject being treated.

Determination of a therapeutically effective amount is well within the capability of those skilled in the art, especially in light of the detailed disclosure provided herein.

For any preparation used in the methods of the invention, the therapeutically effective amount or dose can be estimated initially from in vitro and cell culture assays. Toxicity and therapeutic efficacy of the active ingredients described herein can be determined by standard pharmaceutical procedures in vitro and cell cultures or experimental animals. The data obtained from these in vitro and cell culture assays and animal studies can be used in formulating a range of dosage for use in humans. Such compositions are formulated without undue experimentation. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p. 1.).

Dosage amount and interval may be adjusted individually to levels of the active ingredient which are sufficient to, for example, retard tumor progression in the case of blastic metastases (minimal effective concentration, MEC). The MEC will vary for each preparation, but can be estimated from in vitro data. Dosages necessary to achieve the MEC will depend on individual characteristics and route of administration. Detection assays can be used to determine plasma concentrations.

Depending on the severity and responsiveness of the condition to be treated, dosing can be of a single or a plurality of administrations, with course of treatment lasting from several days to several weeks or until diminution of the disease state is achieved.

The amount of a composition to be administered will, of course, be dependent on the subject being treated, the severity of the affliction, the manner of administration, the judgment of the prescribing physician, etc.

Preferred unit doses of antibody, antibody fraction or antibody conjugate generally comprise from about 0.1 μg immunoglobulin per kilogram body weight to about 100 mg immunoglobulin per kilogram body weight, preferably from about 0.1 μg immunoglobulin per kilogram body weight to about 20 mg immunoglobulin per kilogram body weight, more preferably about 0.1 μg immunoglobulin per kilogram body weight to about 10 mg immunoglobulin per kilogram body weight, and still more preferably about 0.1 μg immunoglobulin per kilogram body weight to about 1.0 mg immunoglobulin per kilogram body weight. Suitable carriers and excipients will vary according to the mode of administration and storage requirements of a composition comprising an antibody, antibody fragment or antibody conjugate.

Compositions of the present invention may, if desired, be presented in a pack or dispenser device, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a-governmental agency regulating the manufacture, use or sale of pharmaceuticals, which notice is reflective of approval by the agency of the form of the compositions or human or veterinary administration. Such notice, for example, may be of labelling approved by the U.S. Food and Drug Administration for prescription drugs or of an approved product insert. Compositions comprising a preparation of the invention formulated in a compatible pharmaceutical carrier may also be prepared, placed in an appropriate container, and labelled for treatment of an indicated condition, as if further detailed above.

In order to facilitate practice of the methods described hereinabove, and/or production of the pharmaceutical compositions described above, the present invention further provides a method of identifying novel drug candidates for treating prior protein related diseases.

The method is effected by screening glycosaminoglycans (GAGs), synthetic polysulfated polysaccharides, heparan sulphate proteoglycans (HSPGs) and pharmaceutical compositions comprising these, and antibodies or fragments of antibodies to GAGs, synthetic polysulfated polysaccharides, HSPGs and prion/doppel/shadoo protein and pharmaceutical compositions thereof for an ability to modulate a prion-glycosaminoglycan complex or prevent formation thereof in mammalian cells. Screening may be effected using the PrP^{Sc} cell free conversion assay or PMCA procedure, or MTT assay. Exemplary screening assays are described in detail in Examples 3, 4 and 8 of the Examples section herein.

Once molecules capable of modulating a protein-glycosaminoglycan complex are identified in vitro further analysis is conducted in order to determine their cell penetration capabilities and their toxicity to mammals. If need be, suitable candidates are modified in order to increase cell penetration thereof and decrease toxicity without substantially affecting their activity in modulating the complex.

Additional, advantages, and novel features of the present invention will become apparent to one ordinarily skilled in the art upon examination of the following examples, which are not intended to be limiting. Additionally, each of the various embodiments and aspects of the present invention as delineated herein above and as claimed in the claims section below finds experimental support in the following examples.

### EXAMPLES

The examples as set forth herein are meant to exemplify the various aspects of carrying out the invention and are not intended to limit the invention in any way.

### Example 1

### Reagents

Antibodies were prepared by immunization of the 8-branched multi-antigenic (MAP) peptides as given in the following sequences: KMMER WEQMCITQ YERESQ (SEQ ID NO: 1), SAMSRPLIHFGSDYEDRYYRE (SEQ ID NO: 2), TNMKHMAGAAAAGAWGGLG (SEQ ID NO: 3), GWGQGGGTHSQWNKPSK, (SEQ ID NO: 4) and RYPPQGGGGWGQPHGGG (SEQ ID NO: 5). Immunization and development of hybridomas and subsequent selection and growth of the monoclonal Ab cell lines were performed by standard procedures familiar to those skilled in the art.

MAP peptides were synthesized by solid phase peptide synthesis (SPPS) with Boc chemistry using procedures familiar to those skilled in the art.

In brief, a four-week old BALB/c mouse was immunized with MAP peptides, mixed with an equal volume of complete Freund's adjuvant. After a few boostings, the titres of tail bleeds from the immunized mice were tested against PrP. Once high titre was observed, the spleens were removed for cell fusion with murine myeloma cells. The hybridoma technique described originally by Kohler and Milstein, Eur. J. Immunol. 6, 511 (1976) has been widely applied to produce hybrid cell lines that secrete high levels of monoclonal antibodies against many specific antigens. Hybridoma supernatants were tested for presence of antibodies specific for prion protein by an ELISA-type assay.

Briefly, microtitre ELISA plates were coated at 37° C. for 1 hour with 1 μg/mL recombinant PrP (100 μL/well). Wells were blocked with 1% BSA in PBS-Tween 20 (PBS-T) and washed with PBS-T. Antibody or cell supernatants diluted in PBS were added and incubated at 37° C. for 1 hour. Plates were washed and alkaline phosphatase conjugated anti-mouse immunoglobulin (100 μL of 1000× dilution) was added for 1 hour at 37° C. After washing, development was byp-nitrophenylphosphate in carbonate buffer and the plate was read at 405 nm.

### Example 2

### Extraction and Purification of HSPGs

The extraction and purification of HSPGs were accomplished in a manner similar to those reported (U.S. Pat. No. 7,094,580, Giuseppetti 1994). Cell preparations were added to cold guanidine extraction buffer (4.0 M guanidine HCl, 0.5 M Na acetate, 10 mM EDTA, 1.0 mM phenylmethylsulfonyl fluoride (PMSF), 100 mM 6-aminohexanoic acid, 5 mM benzamidine HCl, 2% Triton X-100, pH 5.81) and stirred for 24 hr at 4° C. The extracts were centrifuged at 5,500 rpm for 30 min to remove insoluble material and then dialyzed in small pore dialysis tubing (MW cut-off 3,500) into DEAE buffer (50 mM Tris-base, 6.0 M Urea, 0.1 M NaCl, 10 mM EDTA, 10 mM 6-aminohexanoic acid, 1.0 mM PMSF, 0.5% Triton X-100, pH 7.0) and applied to 10 mL DEAE-Sephacel (Sigma Chemical Co., St. Louis, Mo.) columns equilibrated in DEAE buffer containing 0.2% CHAPS (instead of Triton X-100), dialyzed into CHAPS-DEAE buffer, and applied to a high performance liquid chromatography (HPLC) 7.5×7.5 mm TSK DEAE 5PW anion exchange column (Bio-Rad, Richmond, Calif.). Samples were applied at a flow rate of 1 mL/min, and proteins were eluted with a linear gradient from 0.1 M to 0.8 M NaCl. Fractions of 1 ml each were collected and sampled. The salt gradient was monitored by conductivity measurements using a conductivity meter. Comparisons were made to standards of known NaCl concentration in DEAE buffer to calculate the actual salt concentrations of the fractions. The percentage recovery from the HPLC-DEAE columns was 90-95%. Samples were dialyzed into deionized water containing 0.1 mM PMSF and lyophilized. Aliquots were stored at −80° C. and used as indicated.

### Example 3

### Cell-Free Conversion Assay

The cell free conversion reaction can be performed as previously described (Kocisko 1994; Raymond 1997). Purified PrP^{res} is partially denatured with 2.5 M guanidine hydrochloride (Gdn-HCl) for 30 to 60 min at 37° C. An aliquot of 200 ng of HaPrP^{res}, typically 8 mL, is then incubated for 40 hours at 37° C. with about 1 ng immunopurified ³⁵S—PrP^{sen} (about 12,000 cpm/reaction) in a final volume of 20 mL of conversion buffer (50 mM sodium citrate pH 6.5 mM cetyl pyridinium chloride, 0.625% N-lauryl sarcosinate) in the presence or absence of the inhibitory peptides and or antibodies. At the end of the incubation time, each reaction is split 1:10, the major fraction is digested with 100 mg/mL proteinase K (PK) in Tris-saline buffer (50 mM Tris pH 8, 130 mM NaCl) for 1 hour at 37° C. and the minor part (—PK) is reserved as an undigested control.

The PK reaction is stopped by the addition of 10 mL of a mixture of 4 mg/mL thyroglobulin, 20 mM pefabloc to each fraction (+ and −PK). Samples are then precipitated in 5 volumes of methanol and centrifuged for 20 min at 14,000 rpm. The pellets are resuspended in sample buffer (65 mM Tris-HCl pH 6.8, 5% glycerol, 5% SDS, 4 M urea, 5%-mercaptoethanol, 0.5% bromophenol blue), boiled 5 min and analyzed by SDS-PAGE on precast gels. The percent of the conversion is calculated by the ratio between the PK-resistant ³⁵S-labeled bands, and the bands that are approximately 5-10 kDa lower in molecular mass than the non-digested ³⁵S—PrP^{sen}, as quantified by autoradiographic imager analysis.

Various synthetic GAGs and antibodies to the same can be tested for effects on the in vitro conversion of metabolically labeled PrP^{sen} molecules into PrP^{res}. The antibodies and/or GAGs are used in the cell-free conversion assay at a variety of concentrations between 2 ug/mL up to a final concentration of 1 mg/mL. The optimal concentration is about 10 μg/mL.

### Example 4

### Preparation of Tissue Homogenates

Tissue homogenates were prepared according to reported methods (Saa 2006). Healthy and sick animals were perfused with phosphate-buffered saline (PBS) plus 5 mM EDTA prior to harvesting the tissue. Ten percent brain homogenates (w/v) were prepared in conversion buffer (PBS containing 150 mM NaCl, 1.0% Triton X-100, 4 mM EDTA, and Protease Inhibitor Sigma). The samples were clarified by a brief, low speed centrifugation (1500 rpm for 30 s) using an Eppendorf centrifuge. Dilutions of this brain homogenate were done in conversion buffer, and they are expressed in relation to the brain; for example, a 100-fold dilution is equivalent to a 1% brain homogenate.

### PMCA Procedure

Protein Misfolding Cyclic Amplification (PMCA) was performed according to reported methods (Saá 2006). Aliquots of normal and scrapie hamster brain homogenate prepared in conversion buffer were mixed and loaded onto 0.2 mL PCR tubes. Tubes were positioned in a sonicator. Cycles of 60 min of incubation at 37° C. followed by a 60 s pulse of sonication (optionally set at 60% potency) were performed. Samples were incubated without shaking and immersed in the water of the sonicator bath and the entire sonicator was kept inside an incubator at 37° C.

The preferred embodiment of Example 4 involves the use of hamster brain. Other embodiments of the example include the use of mouse or rat brain.

Using the immunoblot or slot-blot assay, amplification of HSPG as stained by heparan sulfate antibody is proposed to be evident at the same time as amplification of PrP^{Sc} (identified by anti-PrP antibody following proteinase K treatment as in Example 3—see Sarafoff et al., J. Biochem. Biophys. Methods, 63 (2005) 213).

### Example 5

### Identification of Tumour Cells that Over-Express Prion Protein

(i) Cells from various tumour cell lines are allowed to grow for 2 days on glass slides incubated in appropriate media at 37° C. in 5% CO₂. After 2 days the media is removed and is replaced with spent culture media containing an anti-PrP antibody to which is conjugated a fluorophore such as rhodamine isothiocyanate. Cells are incubated for a further 5 hours at 37° C. in 5% CO₂. At this time the media is removed and uptake into the cells is assessed by fluorescent microscopy of internalization of the rhodamine fluorophore. Uptake is determined on a relative scale. Alternatively, a fluorescent secondary antibody can be used with a non-conjugated anti-PrP primary antibody (see Example 18).
(ii) Cells from various tumour cell lines were allowed to grow until 70-80% confluent in 6 well plates incubated in RPMI1640 media at 37° C. in 5% CO₂. After 2 days the media was removed, cells were washed once with PBS and then extracted with buffer (NaCl 150 mM, Triton X-100 0.5%, Sodium deoxycholate 0.5%, Tris HCl 5 mM, pH 7.5, 5 mM EDTA). Analysis for PrP was performed using a commercial ELISA kit (SPI Bio) and results normalized to the same protein content (as measured by BCA assay).

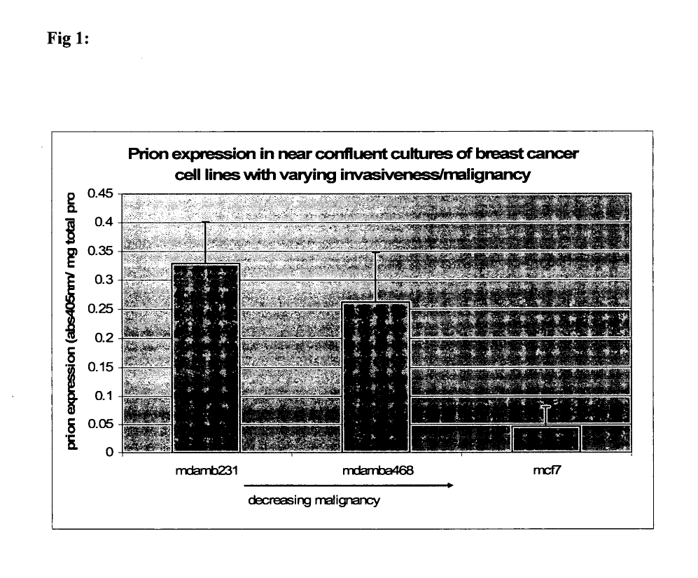

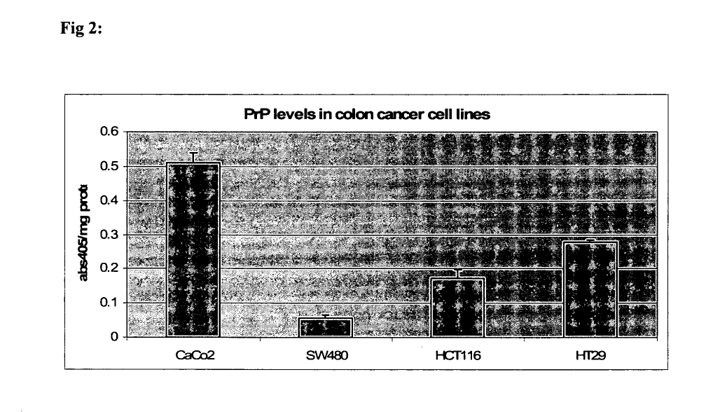

According to the results, more malignant breast cancer cell lines have higher PrP levels (see FIG. 1). Also, colon cell lines that are more malignant have higher PrP levels (see FIG. 2).

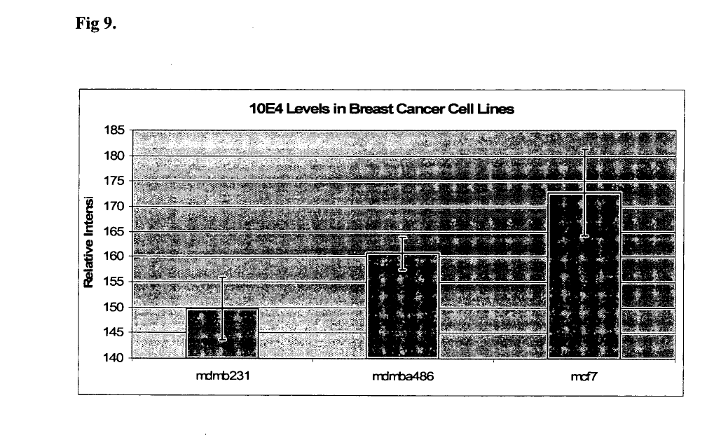

Even more surprising was the discovery of an inverse relationship between PrP levels and HS 10E4 epitope expression (compare FIG. 1 with FIG. 9). Thus the more malignant breast cancer cell lines had lower 10E4 expression (FIG. 9). Analysis of 10E4 expression was performed by slot-blot of cell extracts as described in Example 9.

### Example 6

### Colocalization of Heparan Sulfate and PrP in Prostate Cancer Cells

The prostate cancer cell line 22rvl was obtained from ATCC. The cells in 6 or 12-well multi-well plates were grown on glass coverslips in RPMI1640 medium supplemented with 10% fetal bovine serum, sodium pyruvate, glucose and L-glutamine. At 60-90% confluence cells were washed with cold PBS, then fixed in fresh 2% paraformaldehyde in PBS.

The fixed cells were washed with cold PBS, then blocked by the addition of 2% goat serum in PBS/0.01% Tween-20 30 min rt. The blocked cells were washed with PBS/0.01% Tween-20 then incubated at room temperature for 3 h with primary antibodies diluted 1:200 with PBS/1% BSA. Primary antibodies used were mouse monoclonal IgG anti-human prion (3F4, Covance), and IgM anti-heparan sulfate (clone 10E4, Seikagaku). The monolayers were washed with PBS/0.01% Tween-20 and then incubated with secondary antibodies at 1:2000 dilution. The antibodies used were from Molecular probes: alexaflour 633-conjugated goat anti-mouse IgM and alexafluor 488 conjugated goat anti-mouse IgG. The monolayers were washed with PBS/0.01% Tween-20, then incubated for 2 minutes with DAPI (100 ng/ml in PBS) to visualize nuclei. The cells were washed then mounted in Fluoromount (Southern Biotech).

Stained cells were visualised at 400× magnification using a Leitz Laborlux trinocular compound microscope in fluorescence mode. Images were captured from a ProgRes C10 RGB camera plus (Jenoptik) using ProgRes Capture Pro v2.5 acquisition software. Contrast enhancement of images was performed using Adobe Photoshop CS2 v 9.0. Heparan sulfate and prion protein were found to be co-localized in the cancer cells with both cell surface and endosomal distribution. Co-localization was also evidenced during intercellular transport and cell division.

### Example 7

### Enhanced Killing of HCTl 16 Tumour Cells Treated with Anti-PrP Antibody

Human HCT116 cells were cultured in RPMI1640 medium, harvested and injected (3×10⁶ cells) into nude mice in order to form subcutaneous xenografts. After 8 days a small lump was apparent at the site of subcutaneous injection of the tumour. Mice were then injected intravenously with a dose of 9 mg/kg anti-PrP antibody, twice weekly for two weeks. The tumour was then allowed to continue growing in the mice, and its size determined via a three way measurement using digital calipers. Data was analysed as the average tumour weight of the mice over time.

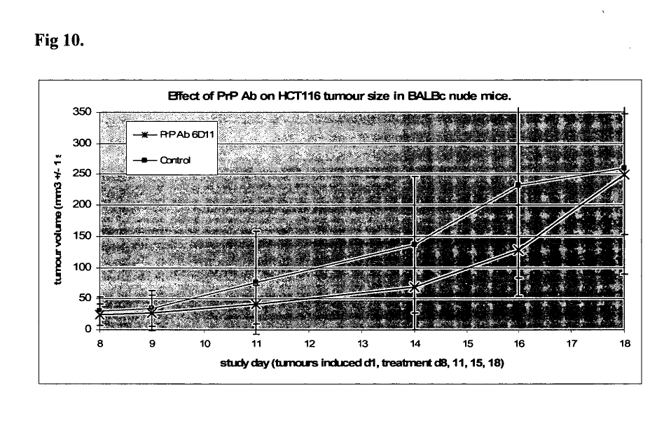

A trend of reduction in tumour growth was seen in the group that received the anti-PrP antibody treatment compared with control animals (FIG. 10) although differences in mean tumour size did not attain significance.

### Example 8

### Effect of PrP Antibodies on Cancer Cell Proliferation Using MTT Assays

Drug sensitivity was evaluated by MTT assay. Cells were plated in 96-well plates (Nunc, Milan, Italy) at a density of 5000 cells/well. After 24 hr, the medium was replaced with fresh growth medium containing 5% FCS and various concentrations of drugs with or without antibody to PrP. After 72 hr of growth in the presence of drugs, cells were assayed for viability. Briefly, MTT reagent (final concentration 500 μg/ml) was added to each well and 4 hr later adherent cells were lysed with 100 μL methyl sulfoxide per well. The absorbance of the formazan product was measured with a Molecular Devices plate reader at a wavelength of 490 nm. Each drug or antibody concentration that produced 50% inhibition of growth (IC50) was estimated by using the relative survival curves. Relative inhibitory rate of cell growth by different concentrations of drug or antibody was calculated according to the following formula: R=(A2−A1)/A2, where R is relative inhibitory rate of cell growth by drug or antibody, A1 is the absorbance value of cells in the presence of drug or antibody for 72 hr and A2 is the absorbance value of control cells without treatment. Each study was performed in triplicate and repeated multiple times. Mouse immunoglobulin was used as a control.

FIG. 3 is a bar graph of the results of screening of PrP antibodies at 10 μg/mL in MTT assay against HCT116 human colon cancer cells (see FIG. 3). The assay determined that there was approximately 100% relative inhibition by BAR221 and BAR236 antibodies. There was also 20-40% relative inhibition by SAF 32, F89/160.1.5, 8H4 and SAF53 antibodies.

FIG. 4 is a line graph of the dose response of selected PrP antibodies in MTT assay against HCT116 human colon cancer cells. Antibodies BAR221 and BAR 236 provided greater than 30% inhibition of tumour cell growth when administered with 1 ug/mL or more. When 5 ug/mL BAR221 or BAR 236 Abs was administered, growth of tumour cells was inhibited by 60%, and administration of 10 μg/mL BAR221 or BAR236Abs provided complete growth inhibition.

### Example 9

### Effect of PrP Antibodies on Cancer Cell Apoptosis

Cells were plated in 96-well plates (Nunc, Milan, Italy) at a density of 50000 cells/well. After 24 hr, the medium was replaced with fresh growth medium without FCS and various concentrations of antibody to PrP. After 24 hr of growth medium was removed and STE-sarkosyl buffer (50 μL) added. Four wells were combined in replicate for protein analyses. Total protein content was measured by BCA assay. Beta-actin, Bcl-2 and 10E4 levels were assessed by slot-blot. Briefly, nitrocellulose membranes were pre-soaked in PBS and 5 μg protein was loaded per slot. The slots were washed twice with 200 uL PBS and the membrane immersed in blocking solution (1% BSA in TEN buffer) for 0.5 h. Primary antibody was added at 20000× dilution in blocking solution for 2 h at room temperature. Primary antibodies were mouse IgM 10E4 (Seikagaku), rabbit polyclonal sc-16323-R to Bcl-2 and rabbit monoclonal to beta-Actin 120-52614 (Santa Cruz). 30% H2O2 added (500 uL to 20 mL) and the solution mixed for 3 min to quench endogenous peroxidase. The membrane was washed in wash buffer (20 mL TEN buffer with 0.1% Tween 20) 3 times for 10 minutes each. Secondary antibody (goat anti rabbit IgG HRP sc-2004 or goat anti-mouse IgM HRP conjugate sc-2064, 20 mL, 20000× dil in blocking buffer) was added for 0.5 h at room temperature. The membrane was washed in wash buffer (20 mL TEN buffer with 0.1% Tween 20) 3 times for 10 minutes each. Transferred to chemiluminescence solution (50:50 peroxide:reagent, 20 mL) for 2 minutes before development. Analysis was performed according to signal intensity by ImageJ software.

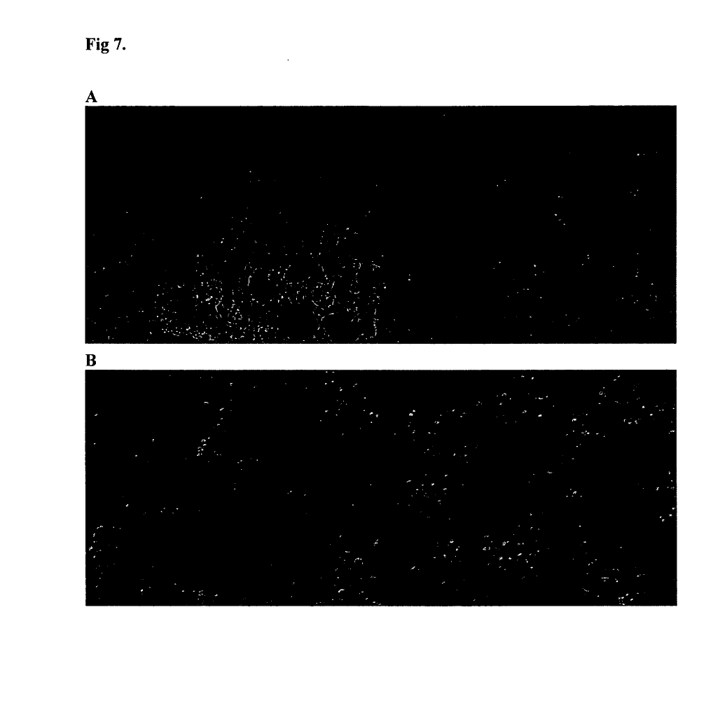

FIG. 7 shows photographs of HCT116 cells grown without (left) and with (right) BAR221 antibody to PrP at 5 μg/mL. A) 50× magnification. B) 200× magnification. Treatment with antibody affords cells with less distinctive morphology—cells become rounder and less adhesive consistent with apoptosis.

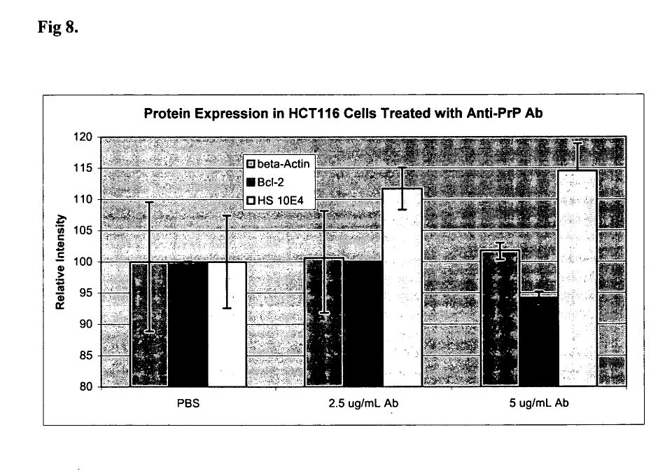

FIG. 8 is a bar graph of slot-blot protein expression data from HCT116 cells treated with different levels of BAR221 PrP antibody. Beta-actin levels remain constant under antibody treatment, confirming similar total protein was loaded onto the membrane. Bcl-2 levels drop statistically at 5 μg/mL PrP antibody indicating increased apoptosis. Conversely, there is a concomitant concentration dependent increase in 10E4 HS expression, suggesting that PrP is involved in regulation of HS expression.

### Example 10

### Enhanced Killing of HCTl 16 Tumour Cells Treated with Combination Therapy

Treatment of HCT116 tumour was found to be more successful upon co-administration of anti-PrP (as disclosed in Example 8) and irinotecan. Briefly, the method of Example 10 was followed except that some groups received a combination therapy of both anti-PrP antibody (9 mg/kg, administered i.v.) and irinotecan (40 mg/kg, administered i.p.). The tumour was then allowed to grow in the mice, and its size determined via a three way measurement using digital calipers. Data was analysed as the average tumour size of the mice over time.

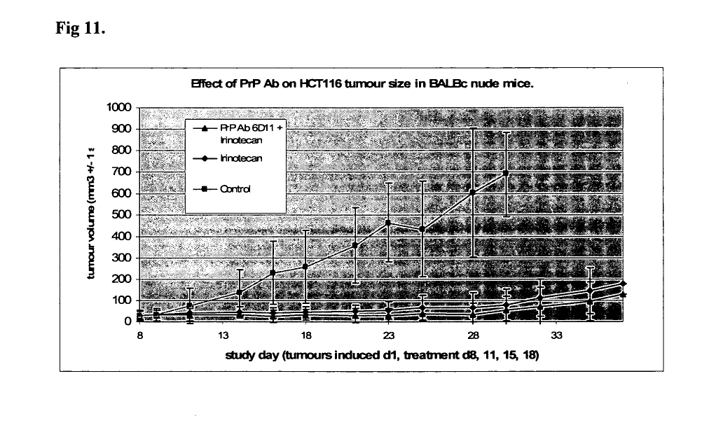

A trend of reduction in tumour size was seen in the group that received the irinotecan/anti-PrP antibody combination treatment over and above the irinotecan alone control group (FIG. 11).

Effect of PrP Antibodies in Combination with IRI on Cancer Cell Proliferation Using MTT Assays

Relative responsiveness to irinotecan treatment upon co-administration of 50 ug/mL 2A5 PrP antibody in MTT assay against various human colon cancer cells. A strong correlation was observed with PrP expression except for HT29, which is known to be irinotecan-resistant.

### Example 11

### Effect of HS Antibodies on Cancer Cell Proliferation Using MTT Assays

Drug sensitivity was evaluated by MTT assay. Cells were plated in 96-well plates (Nunc, Milan, Italy) at a density of 5000 cells/well. After 24 hr, the medium was replaced with fresh growth medium containing 5% FCS and various concentrations of drugs with or without antibody to PrP. After 72 hr of growth in the presence of drugs, cells were assayed for viability. Briefly, MTT reagent (final concentration 500 μg/ml) was added to each well and 4 hr later adherent cells were lysed with 100 μL methyl sulfoxide per well. The absorbance of the formazan product was measured with a Molecular Devices plate reader at a wavelength of 490 nm. Each drug or antibody concentration that produced 50% inhibition of growth (IC50) was estimated by using the relative survival curves. Relative inhibitory rate of cell growth by different concentrations of drug or antibody was calculated according to the following formula: R=(A2−A1)/A2, where R is relative inhibitory rate of cell growth by drug or antibody, A1 is the absorbance value of cells in the presence of antibody for 72 hr and A2 is the absorbance value of control cells without treatment. Each study was performed in triplicate and repeated multiple times.

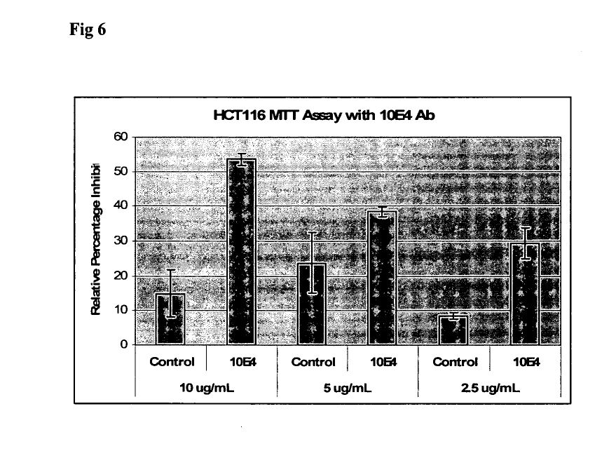

The anti-proliferative response to HS antibody 10E4 as measured in MTT assay against human colon cancer HCT116 cells is depicted in FIG. 6. According to the results there was a significant variance of the relative percentage of inhibition between control cells compared to cells treated with HS 10E4 antibodies. Administration of 10 μg/mL antibody provided 50-60% inhibition of cell growth. Administration of 5 μg/mL antibody provided 35-40% inhibition of cell growth. Administration of 2.5 μg/mL provided 25-30% inhibition.

### Example 12

### Enhanced Killing of WiDr Tumour Cells Treated with Pentosan Polysulfate Combination Therapy

Treatment of WiDr tumour xenografts was found to be more successful upon coadministration of pentosan polysulfate and cytotoxic drug. Briefly, the method of Example 10 was followed except that some groups received a combination therapy of both low molecular weight pentosan polysulfate and cytotoxic drug. The tumour was then allowed to grow in the mice, and its size determined via a three way measurement using digital calipers. Data was analysed as the average tumour weight of the mice over time.

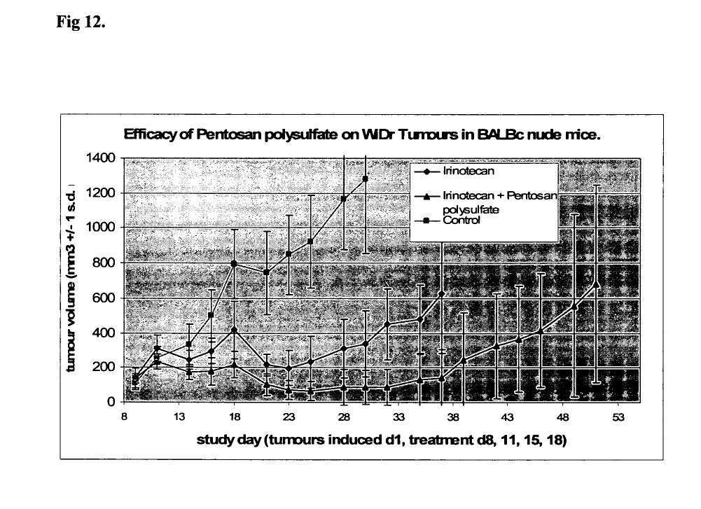

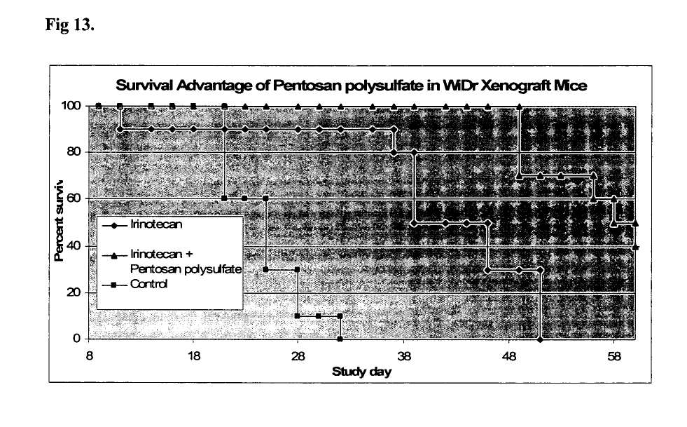

Significant reduction in tumour mass was seen in the animals that received the pentosan polysulfate/drug combination treatments over and above the control group that received drug alone (FIG. 12). Combination therapy with pentosan polysulfate also afforded increased survival time compared to irinotecan alone as is evidenced from the Kaplan-Meier plot (FIG. 13).

### Example 13

### Amplification of specific HSPGs/GAGs

The HSPG to be amplified is isolated and purified from hamsters according to standard procedures known to those skilled in the art. Hamster brain is then treated by heparanase and/or assisted nitric oxide/nitrite cleavage of heparan sulfate chains. Enzymes responsible for such cleavages are treated by addition of inhibitors (such as and not limited to pentosan polysulfate and/or P188 inhibition of heparanase). The purified HSPG is then added to the treated brain homogenate and PMCA is performed with as many cycles as required according to the method outlined in Example 4. The PMCA technique can be enhanced by addition of proteins/enzymes involved in the process of HS synthesis such as PrP, epimerases, sulfotransferases, deacetylases, EXT1 and EXT2 and UDP-sugars (for a more complete (but not limited) list, see Prydz 2000 and Esko 2002). Amplification of HSPG/GAG can be demonstrated by specific immunoblot assays using antibodies to the HSPG according to standard procedures known to those skilled in the prior art.

Amplification of HSPGs/GAGs is also applicable to other species such as, but not limited to, rat and mouse. Such amplification is also extended to use of homogenates of tumours or cultured cells, instead of using brain homogenate. Enhanced amplification of HSPGs/GAGs can potentially be achieved by the supplementation of the media with any of the proteins/enzymes listed in Example 14. A synthetic medium containing PrP and a mixture of the enzymes in Example 14 and other potential cofactors such as vertebrate RNA is also disclosed for amplification of HSPGs/GAGs.

### Example 14

### Mechanism for Amplification of HSPG Messages

A method for the amplification of HSPGs is outlined as follows: in the Golgi which is the site of synthesis of the heparan sulfate chains of HSPGs, PrP/Dpl binds to HSPG.

The HSPG is Gpc-1. The PrP/Dpl-HSPG unit is constrained in a particular conformation dependent upon the sequence of the heparan sulfate chains. It appears that binding of molecules to the N-terminus is able to affect the local conformation within the C-terminal domain of the prion protein. A second PrP/Dpl then binds, which is itself forced into a specific conformation dependent upon the structure of the initial PrP/Dpl-HSPG unit. The second PrP/Dpl molecule, dependent upon its conformation, provides a template for another proteoglycan with ‘stub’ heparan sulfate chains. The chain lengthening/modification of these heparan sulfate chains then takes place in a environment constrained by the PrP/Dpl-HSPG complex such that the new heparan sulfate chains are similar or exact replicas of the chains on the first HSPG. Firstly, a non-sulfated polysaccharide chain precursor is prepared which is then modified by a sequential series of reactions that superimpose complex patterns of sulfation. The sulfation pattern is determined by a complex interplay of sulfotransferases (such as, but not limited to, 3-O-sulfotransferase-1 [3-OST-1], -2, -3A, -3B, -4,6-OST-1, -2, -3 and N-deacetylase/N-sulfotransferase-1 [NDST-I], -2, -3) located at the Golgi. Other enzymes involved in HS synthesis include GaNAc-transferase I, GaNAc-transferase II, GlcAc-transferase II, C-5 GIcA epimerase, 4-OST, 2-OST, EXTL2, EXT1 and EXT2 (See Sasisekharan 2006, Esko 2002 and Prydz 2000).

### Example 15

### In Vivo Infectivity Studies

### 263K Scrapie

Syrian Golden hamsters can be used as an in vivo model of scrapie according to reported methods (Sad 2006). Other animals which may also be used for testing are, for example, mice or rates. Animals should be 4-6 weeks old at the time of inoculation. Anesthetized animals are injected intracerebrally stereotaxically in the right hippocampus with 1 μL of the sample or intraperitoneally with 200 μL of sample. The onset of clinical disease is measured by scoring the animals twice a week using the following scale: 1, normal animal; 2, mild behavioral abnormalities, including hyperactivity and hypersensitivity to noise; 3, moderate behavioral problems, including tremor of the head, ataxia, wobbling gait, head bobbing, irritability, and aggressiveness; 4, severe behavioral abnormalities, including all of the above plus jerks of the head and body and spontaneous backrolls; 5, terminal stage of the disease in which the animal lies in the cage and is no longer able to stand up. Animals scoring level 4 during 2 consecutive weeks are considered sick and are sacrificed to avoid excessive pain using exposure to carbon dioxide.

For control experiments, samples are treated with phosphate buffered saline, heparinase I, heparinase II or heparinase III for 2 hours at 37° C. prior to injection into the animal model. The preferred embodiment is the use of heparinase III.

Animals receiving infectious HSPG will have an average survival significantly less than those animals receiving the heparinase treated HSPG.

### Example 16

### In Vitro Infectivity Studies

In vitro infectivity studies can be performed according to an adapted method (Enari 2006). N2a cells infected with RML mouse-adapted prions are homogenized by passing eight times each through 21- and 25-gauge needles and adjusted to 10% (wt/vol) with IX Dulbecco's phosphate-buffered saline (GIBCO/BRL). After centrifuging for 5 min at 1,000 rpm and at room temperature, supernatants are recovered and stored at −80° C. N2a cells (2-5×10⁴ in 1 mL of medium) are seeded into 24-well plates (Corning Costar) and cultured for 1-2 days before exposure to RML-infected N2a cell homogenate or HSPG extracted from the same, diluted with complete medium. The inoculum is removed after 3 days and the cells are split 1:5 every 3-4 days. After 14 days the cells are assayed for PrP^{Sc} by the cell blot procedure (below).

### Cell Blot Assay for PrP^{Sc}

The assay is performed as described (Enari 2006). In short, cells are transferred to a poly(vinylidene difluoride) membrane, treated with proteinase K, denatured, immunostained with antibody 6H4 (Prionics) followed by horseradish peroxidase-conjugated goat anti-mouse IgG₁, and visualized by enhanced chemiluminescence (ECL kit; Pierce). After exposure, the membrane is stained for 15 min with 0.5 mg/ml ethidium bromide and photographed in UV light to document the transfer of the cell layer.

### Example 17

In another example the infectious material is HSPG isolated from infectious brain homogenate (RML scrapie) or from RML scrapie-bearing N2a cells.

Thus Syrian Golden hamsters are used as an in vivo model of scrapie according to reported methods (Saá 2006). Animals are 4-6 weeks old at the time of inoculation. Anesthetized animals are injected intracerebrally stereotaxically in the right hippocampus with 1 μL of the sample or intraperitoneally with 200 μL of sample. The onset of clinical disease is measured by scoring the animals twice a week using the following scale: 1, normal animal; 2, mild behavioral abnormalities, including hyperactivity and hypersensitivity to noise; 3, moderate behavioral problems, including tremor of the head, ataxia, wobbling gait, head bobbing, irritability, and aggressiveness; 4, severe behavioral abnormalities, including all of the above plus jerks of the head and body and spontaneous backrolls; 5, terminal stage of the disease in which the animal lies in the cage and is no longer able to stand up. Animals scoring level 4 during 2 consecutive weeks are considered sick and are sacrificed to avoid excessive pain using exposition to carbonic dioxide.

For control experiments, samples are treated with phosphate buffered saline, heparinase I, heparinase II or heparinase III for 2 hours at 37° C. prior to injection into the animal model. The preferred embodiment is the use of heparinase III.

Animals receiving infectious HSPG have an average survival significantly less than those animals receiving the heparinase treated HSPG.

### Example 18

This example is similar to Examples 15 and 17, wherein the scrapie strain is 22L or another TSE.

Thus Syrian Golden hamsters are used as an in vivo model of scrapie according to reported methods (see for example Saá 2006). Animals are 4-6 weeks old at the time of inoculation. Anesthetized animals are injected intracerebrally stereotaxically in the right hippocampus with 1 μL of the sample or intraperitoneally with 200 μL of sample. The onset of clinical disease is measured by scoring the animals twice a week using the following scale: 1, normal animal; 2, mild behavioral abnormalities, including hyperactivity and hypersensitivity to noise; 3, moderate behavioral problems, including tremor of the head, ataxia, wobbling gait, head bobbing, irritability, and aggressiveness; 4, severe behavioral abnormalities, including all of the above plus jerks of the head and body and spontaneous backrolls; 5, terminal stage of the disease in which the animal lies in the cage and is no longer able to stand up. Animals scoring level 4 during 2 consecutive weeks are considered sick and are sacrificed to avoid excessive pain using exposition to carbonic dioxide.

For control experiments, samples are treated with phosphate buffered saline, heparinase I, heparinase II or heparinase III for 2 hours at 37° C. prior to injection into the animal model. The preferred embodiment is the use of heparinase III.

Animals receiving infectious HSPG have an average survival significantly less than those animals receiving the heparinase treated HSPG.

In view of the many possible embodiments to which the principles of the invention may be applied, it will be recognized that the illustrated embodiment is only a preferred example of the invention, and should not be understood as a limitation on the scope of the invention. Rather, the scope of the invention is defined by the following claims. We therefore claim as our invention all that comes within the scope and spirit of these claims.

1. A method for the treatment of a prion protein-related disease which comprises administering to a subject in need thereof an effective amount of an agent capable of modulating binding of a prion protein or a prion-like protein to a disease related GAG and/or HSPG.

2. The method of claim 1 wherein the prion protein is PrP^{c}.

3. The method of claim 1 wherein the prion-like protein is a Shadoo or Doppel protein.

4. The method of claim 1 wherein the agent is an antibody or antibody fragment capable of modulating binding of a prion protein or a prion-like protein to a disease related GAG and/or HSPG.

5. The method of claim 1 wherein the agent is an anti-HSPG or anti-GAG antibody, or fragment thereof.

6. The method of claim 1 wherein the agent is an anti-prion protein antibody or an anti-prion-like protein antibody, or fragment thereof.

7. The method of claim 4 wherein the monoclonal antibody is an antibody, or fragment thereof.

8. The method of claim 7 wherein the monoclonal antibody is an antibody to PrP.

9. The method of claim 7 wherein the monoclonal antibody is an antibody to Dpl protein.

10. The method of claim 7 wherein the monoclonal antibody is an antibody to a HSPG or GAG.

11. The method of claim 7 wherein the monoclonal antibody is humanised.

12. The method of claim 7 wherein the fragment is a Fab, Fab′ F(ab′)₂ fragment, or an Fᵥ of said monoclonal antibody.

13. The method of claim 7 wherein the antibody is an anti-inhibitory peptide antibody.

14. The method of claim 7 wherein an antigen recognition domain of the antibody or fragment thereof is encompassed by amino acid sequences as follows:
SEQ ID NO: 1
KMMERVVEQMCITQYERESQ,
SEQ ID NO: 2
SAMSRPLIHFGSDYEDRYYRE,
SEQ ID NO: 3
TNMKHMAGAAAAGAVVGGLG,
SEQ ID NO: 4
GWGQGGGTHSQWNKPSK,
and
SEQ ID NO: 5
RYPPQGGGGWGQPHGGG,.

15. A hybridoma capable of producing the monoclonal antibody of claim 7.

16. The method of claim 1 wherein the modulating agent is administered together with a cytotoxic agent.

17. The method of claim 7 wherein the antibody is conjugated to a cytotoxic agent.

18. The method of claim 1 wherein the modulating agent is selected from the group consisting of a glycosaminoglycan, a synthetic polysulfated polysaccharide, and a heparin sulphate proteoglycan, or a mixture thereto.

19. The method of claim 1 wherein the modulating agent comprises four saccharides UA-GlcN-UA-GlcNAc.

20. The method of claim 1 wherein the modulating agent is a heparin sulfate proteoglycan that is selected from the syndecan or glypican cell surfact proteoglycans, or derived therefrom.

21. The method of claim 1 wherein the modulating agent is a perlecan or serglycin ore derived therefrom.

22. The method of claim 1 wherein the modulating agent is a pentosan polysulfate, preferably xylopyranose polysulfate (XPS) or derived therefrom.

23. The method of claim 1 wherein the modulating agent is an antibody and administered together with a GAG, synthetic polysulfated polysaccharide and/or heparin sulphate proteoglycan and optionally a cytotoxic agent.

24. The method according to claim 1 wherein the modulating agent is a glycosaminoglycan, synthetic polysulfated polysaccharide or heparin sulphate proteoglycan, and is administered together with a cytotoxic agent.

25. An agent capable of modulating binding of prion protein or a prion like protein to a disease related glycosaminoglycan (GAG) or heparan sulphate proteoglycan (HSPG), and is effective for treatment of a prion protein-related disease.

26. An agent according to claim 25 wherein the prion protein is PrPC, or the prion-like protein is a Shadoo or Doppel protein.

27. An agent according to claim 25 wherein the agent is an antibody or antibody fragment capable of modulating binding of a prion protein or a prion-like protein to a disease related GAG and/or HSPG.

28. An agent according to claim 25 wherein the agent is selected from the group of an antibody or fragment thereof to HSPG, GAG, prion protein, and prion-like protein.

29. An agent according to claim 25, wherein (a) the agent is an antibody or antibody fragment capable of modulating binding of a prion protein or a prion-like protein to a disease related GAG and/or HSPG, or (b) the agent is selected from the group of an antibody or fragment thereof to HSPG, GAG, prion protein, and prion-like protein.

30. An agent according to claim 29 wherein the antibody is a monoclonal antibody, or fragment thereof.

31. An agent according to claim 30 wherein the monoclonal antibody is an antibody to PrP, Dpl protein, or to a HSPG or GAG, or wherein the antibody is an anti-inhibitory peptide antibody.

32. An agent according to claim 30 wherein the monoclonal antibody is humanised, or wherein the fragment is a Fab, Fab′ F(ab′)2 fragment, or an Fv of said monoclonal antibody, or wherein the antibody is conjugated to a cytotoxic agent.

33. An agent according to claim 30 wherein an antigen recognition domain of the antibody or fragment thereof is encompassed by amino acid sequences as follows:
SEQ ID NO: 1
KMMERVVEQMCITQYERESQ,
SEQ ID NO: 2
SAMSRPLIHFGSDYEDRYYRE,
SEQ ID NO: 3
TNMKHMAGAAAAGAVVGGLG,
SEQ ID NO: 4
GWGQGGGTHSQWNKPSK,
and
SEQ ID NO: 5
RYPPQGGGGWGQPHGGG,.

34. A hybridoma capable of producing the monoclonal antibody of claim 30.

35. An agent according to claim 25 wherein the modulating agent is administered together with a cytotoxic agent.

36. An agent according to claim 25 wherein the modulating agent is selected from the group consisting of a glycosaminoglycan, a synthetic polysulfated polysaccharide, and a heparan sulphate proteoglycan or a mixture thereof.

37. An agent according to claim 36 wherein the GAG, synthetic polysulfated polysaccharide and/or heparin sulphate proteoglycan is administered together with a cytotoxic agent.

38. An agent according to claim 27, wherein antibody administered together with a glycosaminoglycan, synthetic polysulfated polysaccharide or heparan sulphate proteoglycan, and optionally with a cytotoxic agent.

39. An agent according to claim 25 wherein the modulating agent comprises four saccharides UA-GlcN-UA-GlcNAc.

40. An agent according to claim 25 wherein the modulating agent is a selected from the group of (i) a heparan sulfate proteoglycan that is selected from the syndecan or glypican cell surface proteoglycans, or derived therefrom; (ii) a perlecan or serglycin ore derived therefrom; or (iii) a pentosan polysulfate, preferably xylopyranose polysulfate (XPS) or derived therefrom.

41. An agent according claim 25, wherein the prion protein related disease is selected from the group including a cancer, cancer tumour, amyloid disease, inflammatory condition, or ischemia.


SEQUENCE LISTING (5 sequences)

SEQ ID NO: 1 - protein, 20 aa, Human
KMMERVVEQMCITQYERESQ

SEQ ID NO: 2 - protein, 21 aa, Human
SAMSRPLIHFGSDYEDRYYRE

SEQ ID NO: 3 - protein, 20 aa, Human
TNMKHMAGAAAAGAVVGGLG

SEQ ID NO: 4 - protein, 17 aa, Human
GWGQGGGTHSQWNKPSK

SEQ ID NO: 5 - protein, 17 aa, Human
RYPPQGGGGWGQPHGGG